(19) United States
(12) Patent Application Publication
(10) Pub. No.: US 2008/0195189 A1
(43) Pub. Date: Aug. 14, 2008

(54) DEGRADABLE RESERVOIR IMPLANTS

(75) Inventor: Soheil Asgari, Wiesbaden (DE)

Correspondence Address: DORSEY & WHITNEY LLP;INTELLECTUAL PROPERTY DEPARTMENT, 250 PARK AVENUE, NEW YORK, NY, 10177, US

(73) Assignee: CINVENTION AG, Wiesbaden (DE)

(21) Appl. No.: 12/030,350

(22) Filed: Feb. 13, 2008

Related U.S. Application Data
(60) Provisional application No. 60/889,692, filed on Feb. 13, 2007.

(51) Int. Cl. A61F 2/92; A61F 2/82

(52) U.S. Cl. 623/1.2; 623/1.42

(57) ABSTRACT

Exemplary embodiments of the present invention of a stent are provided, e.g., at least partially biodegradable stent which can have at least one section made of a material having a particular porous structure.

### CROSS-REFERENCE TO RELATED APPLICATION(S)

The present invention claims priority of U.S. provisional application Ser. No. 60/889,692 filed Feb. 13, 2007, the entire disclosure of which is incorporated herein by reference.

### FIELD OF THE PRESENT INVENTION

The present invention relates to medical implants, such as e.g. stents, which can comprise at least one hollow space or lumen within the structural material or structure of the device, other than a pore or pore system, which may be used as a reservoir for a specific amount of active ingredient to be released after implantation into the body.

### BACKGROUND INFORMATION

Implants are widely used as short-term or long-term devices to be implanted into the human body in different fields of applications, such as orthopedic, cardiovascular or surgical reconstructive treatments. Typically, implants are made of solid materials, either polymers, ceramics or metals. To enable drug-delivery, implants have also been produced with porous surfaces or by using porous materials, wherein a drug may be included in the pore system for in-vivo release.

For implantation into body passageways to maintain the patency through the passageways non-degradable and biodegradable materials have been used. Such passageways are for example coronary arteries, peripheral arteries, veins, biliary passageways, the tracheal or bronchial passageways, prostate, esophagus or similar passageways. Typically, implants for such purposes are deployed in different ways, particularly for vascular stents by introducing them percutaneously and positioning the devices to the target region and expanding them. Expansion can be assured e.g. by mechanical means, like balloon or mandrel expansion, or by using super elastic materials that store energy for self-expansion. These implants are designed to keep the lumen of the passageway open and remain as a permanent implant within the body. Typical examples are stents of various structures like, e.g., those described in U.S. Pat. Nos. 4,969,458; 4,733,665; 4,739,762; 4,776,337; 4,733,665, and 4,776,337. Stents are typically made from materials including polymers, organic fabrics and biocompatible metals, such as stainless steel, gold, silver, tantalum, titanium, magnesium and shape memory alloys, such as Nitinol.

Safety and/or efficacy of a stent can be significantly improved by incorporating beneficial agents, for example drugs that are delivered locally. Implants with drug-releasing coatings are, for example, described in U.S. Pat. Nos. 5,869,127; 6,099,563; 6,179,817; and 6,197,051, particularly for stents with drug elution.

Since the amount of drug available in porous structures may be limited, larger drug reservoirs in medical implant structures have been envisaged to increase the drug loading volume. For example, European Patent Application EP 1 466 634 A1 describes a stent design with drug reservoirs by introducing through-holes fillable with a drug either in metallic or polymeric stents by laser cutting, etching, drilling or sawing or the like. The International Patent Publication 96/26682 describes a hollow stent made of a tubular wire, wherein a pharmacological agent may be included inside the lumen of the wire for release through a plurality of openings in the tubular wire.

Japanese Patent Application JP 2005-328893 A describes a stent structure with hollow sections for housing a medicament which may be released through small holes. The hollow structure is produced by a sequence of several deposition and etching procedures.

International Publication WO2004/004602 describes a drug-eluting stent based on a hollow single tube with microscopic lateral holes whereby the tube can retain a therapeutic agent that can be eluted through the multiplicity of pores into a vessel after deployment of the stent. A significant drawback is that a single tube design is mechanically inferior to a more complex, web-like or lattice design of the stent, particularly in terms of radial strength and also longitudinal shortening of the device after implantation.

There is an increasing demand for functional implants that provide a larger available space unit for storage and for delivery of biologically active, pharmacologically active, therapeutically active, diagnostic or absorptive agents into the living organism. Furthermore, there is an increasing demand of using multiple agents or agents that must be available in higher amounts than currently applicable. One of the disadvantages of conventional solutions is that the overall space or free volume available for the uptake of active ingredients in the implant is typically limited. Particularly with coated implants, the amount of active agents is limited either due to the use of polymers or other carriers containing one or more agents, or due to the limited volume that can be provided by the pore system.

One of the objects of the present invention is to overcome the above-described deficiencies.

### SUMMARY OF EXEMPLARY EMBODIMENTS OF PRESENT INVENTION

It is one object of the present invention to provide an implant, preferably a stent, which is at least partially biodegradable in vivo and is capable of releasing active ingredients, such as a drug or a marker or a diagnostic agent etc. A further object of the present invention is to provide an implant design that allows increasing the effective volume of space usable as a reservoir for active ingredients. Another object of the present invention is to provide an implant design that allows providing at least two different lumens usable as reservoirs for active ingredients.

A still further object of the present invention is to provide an implant that can be used as a device for controlled release of active ingredients. Another object of the present invention is to provide multifunctional implants which can be modified in their material properties, particularly the physical, chemical and biologic properties, e.g. biodegradability, x-ray and MRI visibility or mechanical strength. Another object of the present invention is to provide a cardiovascular implant that comprises a hollow, interconnected tubular network as a reservoir for active ingredients. A further object of the present invention is to provide orthopedic, traumatologic or surgical devices, particularly plates, screws, nails, bone grafts, adhesive implants, and the like, that comprise a hollow space as a reservoir for active ingredients.

A further object of the present invention is to provide an implantable device with a compartment as a reservoir for incorporation of beneficial agents. A further object of the present invention is to provide an implantable device as a delivery device for release of biologically active, therapeutically active, diagnostic or absorptive agents, for example for controlled release of biologically active, therapeutically active, diagnostic agents.

A further object of the present invention is to provide an implant for maintaining the patency of body passageways in animals or human beings, for example for maintaining patency of the esophagus, trachea, bronchial vessels, arteries, veins, biliary vessels and other similar passageways, such as a stent.

In an exemplary embodiment of the present invention, an implantable medical device or part thereof can be provided, which may comprise a structure having a plurality of walls, the walls enclosing a lumen for storing at least one active ingredient, wherein at least a part of the walls is made of a material which is biodegradable in-vivo and which can be adapted to allow fluid communication between the lumen and the exterior of the device for releasing the stored ingredient.

In another exemplary embodiment of the present invention, an implantable medical device or part thereof may be provided, which can comprise a structure having a plurality of walls, the walls enclosing a lumen for storing at least one active ingredient, wherein at least a part of the walls is made of a material which is biodegradable in-vivo and which is non-porous and the walls have at least one opening connecting the enclosed lumen with the exterior of the device.

In another exemplary embodiment of the present invention, an implantable medical device or part thereof may be provided, which can comprise a structure having a plurality of walls, the walls enclosing a lumen for storing at least one active ingredient, wherein at least a part of the walls is made of a material which is biodegradable and which is porous having a plurality of interconnected pores.

In a further exemplary embodiment, an implantable device or part thereof may be provided, which can comprise a structure having a plurality of walls, the walls enclosing a lumen for storing at least one active ingredient, wherein at least a part of the walls is made of a material which is biodegradable and which is porous having a porosity (pore volume/total volume of material) 5% to 90%, more preferred from 10% to 80%, and most preferred from 25% to 60%. According to an exemplary embodiment of the present invention, the material structure of at least a part of the walls may have a porosity in the range of about 10 to 90%, preferably about 30 to 90%, most preferably about 50 to 90%, in particular about 60%.

Porosity can mean, but not limited to, the ratio between the net volume of the free available pore space in the material, and the total volume of the material structure excluding the lumen. Porosity may be measured e.g. by a absorption method, such as N₂-porosimetry. The porous material can facilitate a fluid communication between the lumen and the exterior of the device for example for releasing the stored ingredient, which can include pharmacologically, therapeutically, biologically or diagnostically active agent or an absorptive agent.

In the exemplary embodiments of the present invention, substantially all walls or certain sections of the implantable medical device or part thereof may be made of a material which is biodegradable in-vivo, and, preferably a substantially inelastic, e.g., a rigid material.

The device can be, in an exemplary embodiment, for example, a stent adapted for maintaining the patency of at least one of the esophagus, trachea, bronchial vessels, arteries, veins, biliary vessels and other similar passageways, whereas the lumen in the structural material of the stent includes at least one active ingredient. The active ingredient may be configured to be released from the lumen in-vivo, for example in a drug-eluting stent.

In certain exemplary embodiments, the material of the walls can include at least one material which may be biodegradable in-vivo and optionally one of an inorganic material, an organic material, a metal, a ceramic, a polymer or a composite. In other exemplary embodiments, the material of the walls can be completely biodegradable in-vivo.

Furthermore, the device or stent according to an exemplary embodiment of the present invention can be expandable from a contracted state suitable for insertion into a vessel to an expanded state in which the stent supports the surrounding tissue. Optionally, the stent is self-expandable.

In another exemplary embodiment, a stent may be provided, whereas the lumen has an extension in a longitudinal direction of the stent and along a circumference of the stent, which is substantially larger than a radial extension of the lumen.

In a further exemplary embodiment, a stent can be provided, which may comprise a first tube and a second tube concentric to the first tube, wherein the lumen is enclosed between the first and second concentric tube.

In a yet further exemplary embodiment, a stent can be provided, which may comprise a first ribbon helically wound around a tubular space and a second ribbon helically wound around the tubular space corresponding and concentric to the first ribbon, whereas the lumen is enclosed between the first and second concentric ribbons.

In a still further exemplary embodiment, a stent can be provided, whereas the stent may be formed by a plurality of hollow annular elements each having a sub-lumen, which annular elements are arranged such that each annular element circumferences a tubular space and each annular element has a different inclination from an adjacent abutting annular element, wherein adjacent annular elements are joined at an abutting location to form a passage between two abutting annular elements. Optionally, the annular elements can comprise openings facing the exterior of the tubular space.

In another exemplary embodiment, a stent can be provided, whereas the stent may be formed of a brick wall structured mesh of hollow struts, whereas continuous struts extend in a longitudinal direction, which are connected by linking struts. Optionally, the brick walled structure totally circumferences a tubular space, such that the brick walled structure repeats periodically and perpetually along the circumference.

In still another exemplary embodiment, a stent may be provided, whereas the stent can be formed by a plurality of hollow annular wave elements each having a sub-lumen, which annular wave elements are arranged such that each annular element circumferences a tubular space and each annular element abutting an adjacent annular element, whereas adjacent annular elements are joined at an abutting location to form a passage between two abutting annular elements. Optionally, the tubular space has a shape of a bifurcated tube.

According to an exemplary embodiment of the present invention, a part of the implantable device or stent has a form selected from a ring, a torus, a hollow cylinder segment, a tube segment, a helical, a brick walled and/or a web structure, whereas all these structural parts can include at least one lumen enclosed by walls of porous materials.

According to another exemplary embodiment of the present invention, the lumen of the implantable device or stent may have a volume from about 50 nanoliter (nl) to 100 milliliter (ml), preferably from about 1 microliter (μl) to 10 ml, and more preferably from about 150 μl to 500 μl.

Composing an exemplary embodiment of the implant or stent from the group of standard forms or parts as described above may allow an effective manufacturing of a wide variety of stent shapes having large lumen or reservoirs for storing active ingredients, also in case the stents should be custom made.

The present invention will now be described in greater detail with reference to the preferred embodiments illustrated in the accompanying drawings. The following description makes reference to numerous specific details in order to provide a thorough understanding of the present invention. However, each and every specific detail needs not to be employed to practice the present invention.

These and other objects, features and advantages of the present invention will become apparent upon reading the following detailed description of embodiments of the present invention.

### BRIEF DESCRIPTION OF THE DRAWINGS

Further objects, features and advantages of the present invention will become apparent from the following detailed description taken in conjunction with the accompanying figures showing illustrative embodiments of the present invention, in which:

FIG. 1 is an illustration of a tubular stent structure having an inner lumen according to an exemplary embodiment of the present invention;

FIG. 2 is an illustration of a helical stent structure whereas the material encloses a lumen in the structural material itself according to a further exemplary embodiment of the present invention;

FIG. 3 is an illustration of a ring-segmented stent structure according to a further exemplary embodiment of the present invention;

FIG. 4 is an illustration of a wall/brick structured stent structure according to a further exemplary embodiment of the present invention;

FIG. 5 is an illustration of a variant of strut forms for a stent structure according to a further exemplary embodiment of the present invention;

FIG. 6 is an illustration of a punched pattern for a stent structure according to a further exemplary embodiment of the present invention;

FIG. 7 is an illustration of a web pattern for a stent structure according to a further exemplary embodiment of the present invention;

FIG. 8 is an illustration of an interconnected woven pattern for a stent structure according to a further exemplary embodiment of the present invention;

FIG. 9 is an illustration of a bifurcated tube of a stent structure according to a further exemplary embodiment of the present invention; and

FIG. 10 is an illustration of a cross section of a bifurcated tube of a stent structure according to a further exemplary embodiment of the present invention.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention.

### DETAILED DESCRIPTION OF EXEMPLARY EMBODIMENTS

The term “biodegradable” as used herein can include but not limited to any material which can be removed in-vivo, e.g., by biocorrosion or biodegradation. Thus, any material, e.g., a metal or organic polymer that can be degraded, absorbed, metabolized, or which is resorbable in the human or animal body may be used either for a biodegradable metallic layer or as a biodegradable template in the exemplary embodiments of the present invention. In addition, as used herein, the terms “biodegradable”, “bioabsorbable”, “resorbable”, and “biocorrodible” are can encompass but not limited to materials that are broken down and may be gradually absorbed or eliminated by the body in-vivo, regardless whether these processes are due to hydrolysis, metabolic processes, bulk or surface erosion.

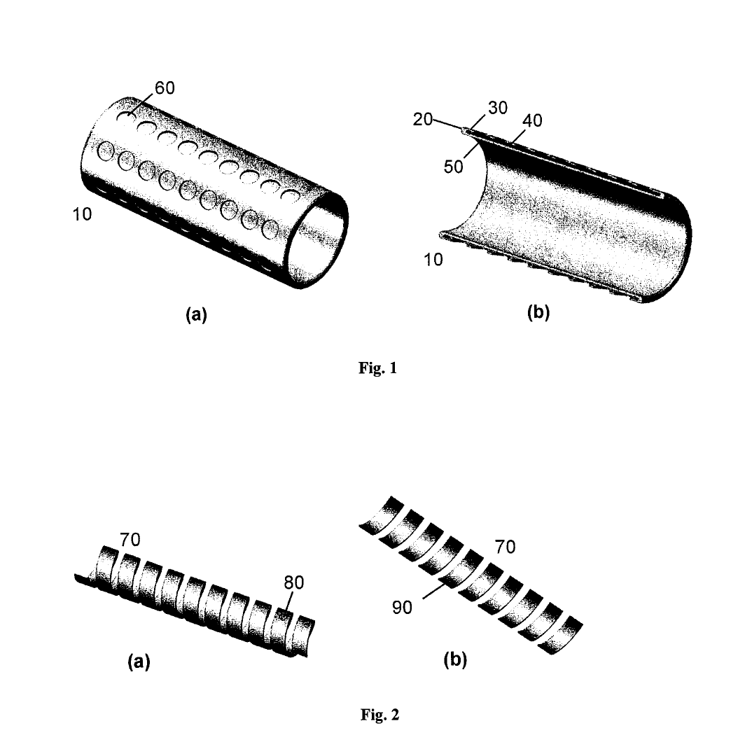

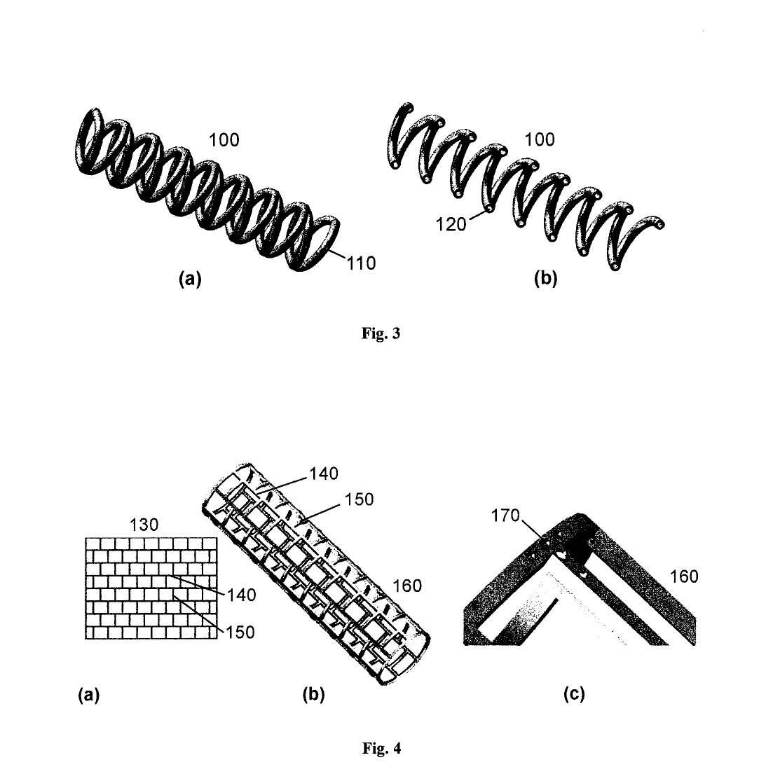

The terms “lumen”, “compartment” or “reservoir” can be used herein to describe (but not limited to) a an essentially closed hollow space, other than a pore or pore system, enclosed by walls of the implant material. Examples of a lumen are shown, e.g., in FIG. 1b, whereas the lumen is enclosed between a first and a second concentric tube, in FIG. 2b, wherein the lumen is enclosed between a first and a second concentric ribbon, or in FIG. 3b, whereas the lumen is inside a hollow double helical structure and may either be continuous or discontinuous, i.e. a plurality of not interconnected reservoirs.

The term “porous” as used herein can designate, but not limited to, a property of a material, which is determined by the presence of a plurality of interconnected pores. The volume of the pores can be assessed by measuring the porosity of the material as further described herein. “Porous” generally may not include holes like boreholes or the like.

The terms “active ingredient”, “active agent” or “beneficial agent” as used herein can include but not limited to any material or substance which may be used to add a function to the implantable medical device. Examples of such active ingredients can include biologically, therapeutically or pharmacologically active agents, such as drugs or medicaments, diagnostic agents, such as markers, or absorptive agents. The active ingredients may be a part of the first or second particles, such as incorporated into the implant or being coated on at least a part of the implant. Biologically or therapeutically active agents may comprise substances being capable of providing a direct or indirect therapeutic, physiologic and/or pharmacologic effect in a human or animal organism. A therapeutically active agent may include a drug, pro-drug or even a targeting group or a drug comprising a targeting group. Examples for biologically active ingredients may include living cells or tissue, microorganisms, such as bacteria, fungi, algae, virus; enzymes, vectors, targeting-groups etc. An “active ingredient” according to exemplary embodiments of the present invention may further include but not limited to a material or substance which may be activated physically, e.g., by radiation, or chemically, e.g., by metabolic processes.

Without being bound to any particular theory, with the exemplary embodiments of the present invention, implants may be provided which can be at least partially biodegradable in-vivo and which may comprise substantially larger volumes of space which may be used as a reservoir for active ingredients. For example, the exemplary implants can comprise at least one hollow space or lumen within the structural material or structure of the device, other than a pore or pore system, which may be used, e.g., as a reservoir for a specific amount of active ingredient to be released after implantation into the body.

In one exemplary embodiment, the implant can comprise a tubular structure. The tubular structure may comprises in its longitudinal axis an inner lumen, whereby the inner wall may be closed, and the outer wall of the cylindrical tube may comprise at least one opening or a plurality of openings. Between both walls, the stent can comprises an inner compartment, or respectively a reservoir. At least a part of one of the walls may be adapted to allow fluid communication between the inner lumen and the exterior of the stent walls. For example, the inner wall may be porous, e.g. to allow elution of an active agent into the inner hollow space of the cylindrical tube, or the outer wall may be porous, e.g., to allow elution of an active agent from the lumen between both walls to the exterior space of the cylindrical tube.

FIG. 1a shows an exemplary embodiment according to the present invention of an implant or stent 10 with a tubular or essentially cylindrical structure. A cross-sectional view of the implant 10 is shown in FIG. 1b. The exemplary tubular structure comprises in its longitudinal axis an inner lumen 20, whereby the inner wall 50 can be closed, and the outer wall 30 of the cylindrical tube may comprise at least one opening 60 or a plurality of openings. Between both walls, the stent can comprises an inner compartment 40, or respectively a reservoir.

The size of the exemplary reservoir or lumen may be adjustable, e.g., by selecting the dimensional sizes of length and width and diameter, and/or also by appropriate selection of the distance between the inner and outer wall that define the inner lumen or respective reservoir of the implant. A further exemplary embodiment of the present invention can be that the tubular structure may comprise at least one opening or a plurality of openings either on the outer surface or the inner surface of the tube or, in further embodiments on both surfaces in any combination. The openings can have a round shape, ellipsoid shape, rectangular shape or any other regular or irregular geometry or any combination thereof. The openings can further improve the release of biologically active, therapeutically active, diagnostic or absorptive agents or any combination thereof. Furthermore, the openings may further allow for the absorption of compounds in physiologic fluids into the compartment.

One having ordinary skill in the art can determine the appropriate option in terms of dimension and embodiment of porous compartments and openings, if any, depending on the target area with the body of the living animal or human being. For example, an exemplary embodiment for use as an artery or vein graft must have appropriate dimensions for implanting the device. Furthermore, an intended release of a therapeutic agent locally to the surrounding vessel wall may further require appropriate dimensions of the pores or openings to sufficiently absorb substances or to release the beneficial agents. The use for a systemic or local organ oriented release of the beneficial agents may require porous walls either at the inner wall of the stent, or at the outer wall, or both.

A person skilled in the art can easily determine the appropriate option in terms of dimension and embodiment of openings depending on the target area with the body of the living animal or human being. For example, in one exemplary embodiment for use as a tracheal or bronchial stent the implant must have appropriate dimensions for implanting the device. Furthermore, the intended release of a therapeutic agent locally to the surrounding tissue requires openings at the outer surface. With the exemplary use for a systemic or local organ oriented release of beneficial agents, it may be appropriate to comprise openings at the inner surface to enable the release of those agents into the lumen where body fluids, e.g., blood, are present.

In another exemplary embodiment, the implant can be a stent comprising a helical tube of a band-like or stripe-like structure. The helical structure is such-like that a flexible distortion is principally possible due to the design. The band-like or stripe-like structure may be hollow and comprises an inner lumen or reservoir, which is enclosed by walls. At least a part of the walls is adapted to allow for a fluid communication between the inner lumen and the outside of the stent structure. The exemplary structure may additionally comprise at least one opening.

FIG. 2a shows an exemplary embodiment of a possible stent structure 70 according to the present invention which can comprise a helical tube of a band-like or stripe-like structure. A cross-sectional view of the implant 70 is shown in FIG. 2b. The band-like or stripe-like structure is hollow and comprises an inner lumen or reservoir 90. The structure may also optionally comprise at least one opening 80.

In another exemplary embodiment, the implant can comprise a double helical structure of interconnected, helically winded tubes. The double helical structure may be hollow and comprises a continuous inner lumen or respective reservoir, or a plurality of reservoirs. In one optional exemplary embodiment, the helical tubular stent can comprise more than two helices.

FIG. 3a shows another exemplary embodiment an implant, e.g., a stent 100 having a double helical structure of optionally interconnected, helically winded tubes. The exemplary structure may additionally comprise at least one opening 110. The cross-sectional view of the implant provided in FIG. 3b illustrates that the double helical structure is hollow and may comprise a continuous inner lumen 120. In an alternative exemplary embodiment, the lumen may be discontinuous, e.g., a plurality or not interconnected reservoirs 120.

In further exemplary embodiments the helical stripe (as shown in FIGS. 2a and 2b) or the at least one helical tube (as shown in FIGS. 3a and 3b) may comprise peaks or serpentines, either symmetrically or asymmetrically, or any desired pattern of peaks and/or serpentines. In addition, a plurality of peaks and/or serpentines may be embedded in any desired combination, whereby also the angles and radius can be different. Furthermore, the peaks and serpentines can be of rectangular shape, either with rounded or without rounded edges of the struts. The struts can have different width and/or depth, i.e. aspect ratios, at different sections along their structures. In some embodiments it can be preferred to have combination of rectangular or rounded peaks and/or serpentines or any combination thereof.

In another exemplary embodiment, the implant or stent can be composed of a mesh-like tube or lattice. One exemplary embodiment of the implant or the stent can comprise a rectangular pattern in a two-dimensional view.

FIG. 4a shows an exemplary embodiment of a rectangular pattern 130 according to the present invention in a two-dimensional view. The exemplary lattice structure comprises in longitudinal direction continuous struts 140 that are connected by linking struts 150. The lattice 130 may be formed to a tubular implant 160 as shown in FIG. 4b. The struts 140 and 150 can be hollow and comprise an interconnected inner compartment or reservoir, or, alternatively, a plurality of discrete reservoirs. At least a part of the strut walls may be made of a porous material as defined herein. The structure may also comprise at least one or a plurality of openings 170 as illustrated in FIG. 4c, which is a magnification of a section of FIG. 4b.

The exemplary lattice structure can comprises in longitudinal direction continuous struts that are connected by linking struts. The lattice can be formed to a tubular, cylindrical stent as described in the drawings. The struts may be hollow and comprise an interconnected reservoir. In certain exemplary embodiments, the structure may also comprise at least one or a plurality of openings.

For example, in one specific exemplary embodiment for use as a coronary or peripheral stent, the implant should have appropriate dimensions for implanting the device. The angle between one linking strut and the continuous struts can be about 90°, in other exemplary embodiments, the angle can be modified to any preferable pattern with angles from about 0.1° to 179°. The porous lattice tube may, e.g. comprise at least two continuous struts that are linked. The number and distance of continuous and linking struts can be varied according to the intended mechanical properties, the required volume of the porous compartment or respective reservoir. In addition, the orientation of the linking struts can be varied. Furthermore, an asymmetric design of linking struts, e.g., identical numbers and/or orientation and/or distances and/or angles, may be used or asymmetric designs with different numbers and/or orientations and/or distances and/or angles. Particularly for expandable stents it may be desirable to select an exemplary embodiment that can be appropriate, whereby a person skilled in the art can easily identify the appropriate design e.g. by using finite element analysis to determine the optimal configuration. The thickness of the struts can play an important role for elastomechanical properties of the implant. For expandable devices, but not limited to strut thicknesses in a range of about 10 μm up to 500 μm, more preferable from about 50 μm to 400 mm and even more preferable from about 70 μm to 200 μm may be used. The thickness can be larger or smaller, depending on the requirements of the implant regarding mechanical or biomechanical stress occurring after implantation. For example, a person skilled in the art can select larger thicknesses for implants that are used as peripheral stents for arteries in the knee or below the knee.

In addition, the aspect ratio, e.g., the ratio between width and depth of a strut, may be varied as appropriate. In certain exemplary applications that utilize a low profile struts with lower depth may be used. Therefore, the aspect ratios can be in a range from about 20:1 to 1:20, such as from about 10:1 to 1:10 or from about 2:1 to 1:2.

The drawings illustrate the basic aspects of the exemplary embodiments of the present invention and are not limited to any of the aforesaid aspects. For example, the edges of the struts can be rounded. In some exemplary embodiments, for example, in order to increase the overall surface or to optimize the stress distribution for expandable implants, serpentines and peaks may be embedded into the struts. For example, the linking struts may comprise at least one peak or one serpentine with two peaks. The orientation of the peaks or serpentines can be varied, e.g., a left-hand oriented peak or right-hand oriented serpentine with a right-hand oriented peak first and a right-hand oriented peak second or vice versa. In certain exemplary embodiments, the modified linking struts may all have the same design; in other exemplary embodiments, the struts can have alternating patterns or any different pattern or combination thereof. In further exemplary embodiments, the continuous struts may comprise peaks or serpentines, either symmetrically or asymmetrically, or both the continuous struts and the linking struts may comprise any desired pattern of peaks and/or serpentines. The exemplary design is not limited to one peak or one serpentine, it is also possible to embed a plurality of peaks and/or serpentines in any desired combination, whereby also the angles and radius can be different.

The length of the exemplary stents as described herein can be dependant on the intended use of the stent, e.g. in a range of about 100 micrometer (μm) to 100 centimeter (cm), such as from about 1000 μm to 10 cm, or from about 5 millimeter (mm) to 60 mm, or even from about 7 mm to 40 mm. The diameter can be selected e.g. in a range from about 5 nanometer (nm) to 20 cm, such as from about 1000 nm to 10 cm, or from about 500 μm to 10 mm, or even from about 500 μm to 10,000 μm. Furthermore, the ratio of length to width of the stent tube can be selected from about 20:1 to 10:1, more preferable from about 8:1 to 5:1 and even more preferable from about 4:1 to 2:1. The exemplary ratio may depend on the intended use of the implant and the capacity of the reservoir compartment.

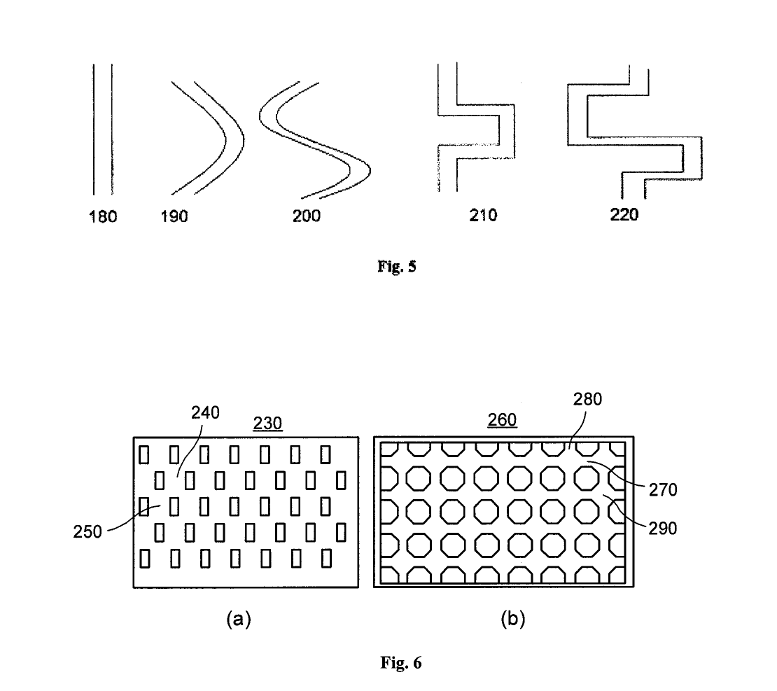

FIG. 5 illustrates exemplary embodiments of several possible strut forms. For example, the edges of the strut can be rectangular 180, the edges of the strut can be rounded 190 or a serpentine can be embedded into the strut 200. The strut can comprise at least one peak 210 or one serpentine with two peaks 220. The orientation of the peaks or serpentines can be varied, e.g. a left-hand oriented peak or right-hand oriented serpentine with a right-hand oriented peak first and a right-hand oriented peak second or vice versa.

The peaks and serpentines can be of rectangular shape, either with rounded or without rounded edges of the struts. Furthermore, the struts can have different width and/or depth, i.e. aspect ratios, at different sections along their structures. In some embodiments it can be preferable to have a combination of rectangular or rounded peaks and/or serpentines or any combination thereof.

In another exemplary embodiment, the open cells, e.g., the space between the struts, of the above described exemplary structure may comprise the struts and the struts comprise the open cells. Therefore, this specific embodiment has to be seen as a “negative” of the aforesaid embodiment.

FIG. 6a shows a open cell pattern 230 in a two-dimensional view. The lattice structure comprises narrow continuous struts 240 connected by broader linking struts 250. FIG. 6b displays a pattern in which the continuous struts 270 and linking struts 280 comprise nodes 290 at their intersections.

In this exemplary embodiment, the continuous struts and linking struts comprise nodes at their intersections. The nodes can have different geometric shapes and dimensions. Particularly, the distances between the nodes, distances of linking struts and the segments of continuous struts between the nodes can be modified similar to the above described embodiments. Hence, also the modification of continuous struts and linking struts can be embedded as explained above.

In another exemplary embodiment, the porous implant can be a mesh-like tube with a rhombic shape of the open cells. The struts are porous and comprise an interconnected inner porous compartment or respective reservoir. The structure may also comprise at least one opening.

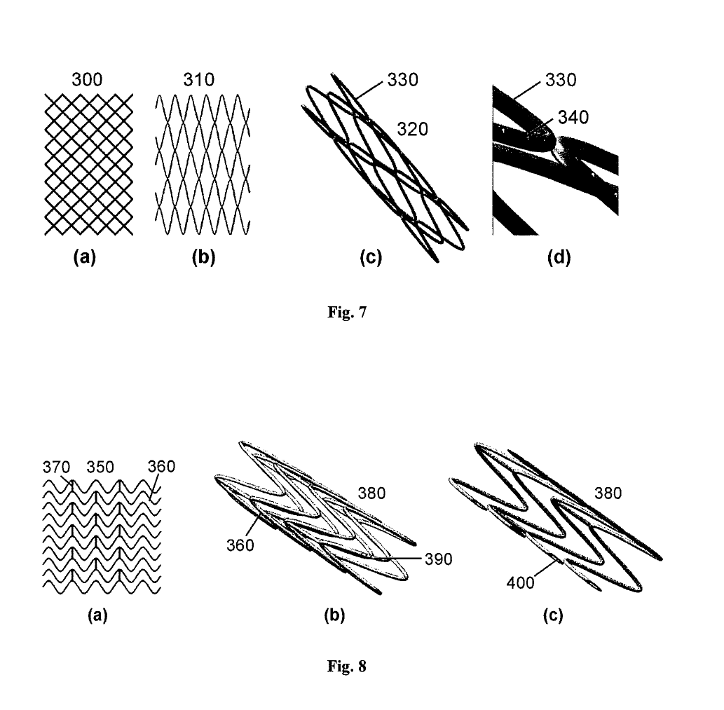

FIG. 7a and FIG. 7b show exemplary embodiments of mesh-like patterns in a two-dimensional view, wherein the open cells have a square shape 300 and a rhombic shape 310, respectively. The mesh 310 can be formed to a tubular implant 320 comprising a mesh-like tube with a rhombic shape of the exemplary open cells as illustrated in FIG. 7c. The struts 330 can be optionally hollow, and comprise an interconnected inner compartment or respective reservoir. The structure may also comprise at least one opening 340 as shown in FIG. 7d, which is a magnification of a section of FIG. 7c.

The angle between the struts in the longitudinal axis may be about 30° to 90°, and the angle can be modified to any preferable pattern with angles from about 0.1° to 179°. According to another exemplary embodiment of the present invention, the angle between the struts in the rectangular axis is about 20° to 120°. The struts form at their intersections a node, whereby at least two nodes are comprised. The exemplary implant can comprise a segment between two nodes, hence, at least one segment can be included. The struts between the nodes may be linking struts. The number and distance of nodes and linking struts can be varied according to the intended mechanical properties, the required volume of the porous compartment or respective reservoir. In addition, the orientation of the linking struts can be varied. An asymmetric design of linking struts may also be used, i.e. identical numbers and/or orientation and/or distances and/or angles. Particularly for expandable implants it is desirable to select an exemplary embodiment that is appropriate, whereby a person skilled in the art can easily identify the appropriate design e.g. by using finite element analysis to determine the optimal configuration. The thickness of the struts can play an important role for elastomechanical properties of the implant. Strut thickness may be as described above.

Further, the aspect ratio, e.g., the ratio between width and depth of a strut, may be selected as described above.

In another exemplary embodiment, the porous implant or stent can comprise a tube with a parallel lattice with interconnecting links. The struts are porous and comprise an interconnected porous compartment or respective reservoir. In specifically preferable embodiments, the structure also comprises at least one opening or a plurality of openings.

FIG. 8a shows an exemplary embodiment of an undulated lattice 350 according to the present invention in a two-dimensional view, wherein the parallel, undulated struts 360 are interconnected by linking struts 370. The exemplary lattice 350 may be formed to a tubular implant 380 as illustrated in FIG. 8b. The structure may comprise at least one opening 390. The cross-sectional view of the implant 380 illustrated in FIG. 8c shows that the structure may optionally be hollow, and comprises an interconnected inner compartment 400 or respective reservoir.

In the longitudinal axis, at least two continuous struts are interconnected by at least one linking strut. The length and diameter of the implant can be in a range as described above.

The porous compartment allows for the incorporation or release of beneficial agents, preferably biologically active, therapeutically active, diagnostic or absorptive agents or any combination thereof. Furthermore, the porous compartment can also facilitate the absorption of compounds in physiologic fluids into the compartment. One having ordinary skill in the art can determine the appropriate option in terms of exemplary dimension and exemplary embodiment of openings depending on the target area with the body of the living animal or human being. For example, in one exemplary embodiment for use as a biliary or coronary stent, the implant must have appropriate dimensions for implanting the device. The angle between one linking strut and the continuous struts is about 10° to 160°, but the angle can be modified to any preferable pattern with angles from about 0.1° to 179°. The number and distance of continuous and linking struts can be varied according to the intended mechanical properties, the required volume of the porous compartment or respective reservoir. The continuous struts may comprise a symmetric or asymmetric pattern of wave-like peaks, whereby the orientation of the peaks can be alternating or non-alternating. The angle of the peaks can be varied from about 10° to 179°, such as from about 15° to 160°, or from about 25° to 120°. In addition, the orientation of the linking struts can be varied. Furthermore, in specific embodiments it is required to have asymmetric design of linking struts may be used, i.e. identical numbers and/or orientation and/or distances and/or angles.

The design of different porous implants is not limited to the above described basic geometric embodiments. For example, implants may also have a combined geometry of the tube, i.e. bifurcated tube at one or more sides or at one lateral end or at both lateral ends and any combination thereof. It could be preferable to implant stents or stent grafts into bifurcated vessels for example, therefore it is useful to have an implant design that follows the natural anatomy of the targeted organ, organ structure or organ vessel.

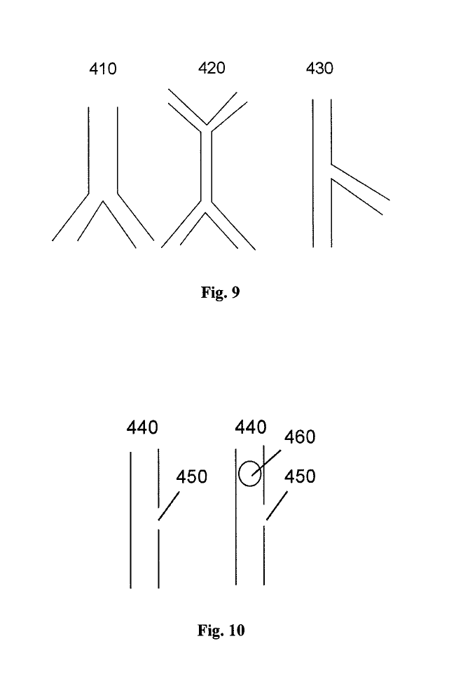

FIG. 9 illustrates exemplary embodiments of three options for implant designs according to the present invention. The implants can have a combined geometry of the tube, e.g., bifurcated tube at one 430 or more sides or at one lateral end 410 or at both lateral ends 420. The implants can have different [ ] diameters at the ends or at any section of the implant as shown in FIG. 9.

Moreover, the implants or stents may have different diameters at the ends or at any section of the implant, e.g. to address the anatomy of target vessels that have a narrowing profile. Another exemplary embodiment comprises at least one cut out within the structure, e.g. for use in bifurcating vessels or complex anatomical structures. The implants may be used in combination, e.g. to allow the implantation of stent into a bifurcation area of arteries or veins.

FIG. 10 shows an exemplary embodiment of an implant 440 according to the present invention comprising a cut out 450 within the structure. The implant 440 can also have a bifurcated tube at one 460 or more sides.

In another exemplary embodiment the open cells, e.g., the space between the struts, of the previously described exemplary embodiment can comprise the struts and the struts comprise the open cells. Therefore, this exemplary embodiment can be seen as a “negative” of the previously-described exemplary embodiment. In this exemplary embodiment, the linking struts comprise large nodes at their intersections. With regard to the specific exemplary aspects of these exemplary embodiments, the nodes can have different geometric shapes and dimensions. For example, the distances between the nodes, distances of linking struts and the segments between the nodes can be modified similar to the aforesaid embodiments. Hence, the modification of continuous struts and linking struts can be embedded as described above.

Exemplary Materials

Any suitable implant material may be used in the manufacture of the implants of the exemplary embodiments of the present invention, with the preference that at least a part of the implant walls comprises a material that is biodegradable, such as biodegradable metals which can include, e.g., metals, metal compounds, such as metal oxides, carbides, nitrides and mixed forms thereof, or metal alloys, e.g. particles or alloyed particles including alkaline or alkaline earth metals, Fe, Zn or Al, such as Mg, Fe or Zn, and optionally alloyed with or combined with other particles selected from Mn, Co, Ni, Cr, Cu, Cd, Pb, Sn, Th, Zr, Ag, Au, Pd, Pt, Si, Ca, Li, Al, Zn and/or Fe. In addition suitable are, e.g., alkaline earth metal oxides or hydroxides, such as magnesium oxide, magnesium hydroxide, calcium oxide, and calcium hydroxide or mixtures thereof. In further exemplary embodiments, the biodegradable metal-based particles may be selected from biodegradable or biocorrosive metals or alloys based on at least one of magnesium or zinc, or an alloy comprising at least one of Mg, Ca, Fe, Zn, Al, W, Ln, Si, or Y. Furthermore, the implant may be substantially completely or at least partially degradable in-vivo. Examples for suitable biodegradable alloys comprise e.g. magnesium alloys comprising more than 90% of Mg, about 4-5% of Y, and about 1.5-4% of other rare earth metals, such as neodymium and optionally minor amounts of Zr; or biocorrosive alloys comprising as a major component tungsten, rhenium, osmium or molybdenum, for example alloyed with cerium, an actinide, iron, tantalum, platinum, gold, gadolinium, yttrium or scandium.

The metal or metal alloy may include in an exemplary embodiment, e.g.:
(i) 10-98 wt.-%, such as 35-75 wt.-% of Mg, and 0-70 wt.-%, such as 30-40% of Li and 0-12 wt.-% of other metals, or
(ii) 60-99 wt.-% of Fe, 0.05-6 wt.-% Cr, 0.05-7 wt.-% Ni and up to 10 wt.-% of other metals; or
(iii) 60-96 wt.-% Fe, 1-10 wt.-% Cr, 0.05-3 wt.-% Ni and 0-15 wt.-% of other metals;
   whereas the individual weight ranges can be selected to always add up to 100 wt.-% in total for each alloy.

In such exemplary embodiments, the implant can be mainly degraded to hydroxyl apatite within the living body. This property of the exemplary implant material can be especially advantageous for implants with a temporary function.

In other exemplary embodiments, the material particles may include biodegradable organic materials, for example—without excluding others—collagen, albumin, gelatine, hyaluronic acid, starch, cellulose (methylcellulose, hydroxypropylcellulose, hydroxypropylmethylcellulose, carboxymethylcellulose-phtalate); furthermore casein, dextrane, polysaccharide, fibrinogen, poly(D,L lactide), poly(D,L-lactide-Co-glycolide), poly(glycolide), poly/hydroxybutylate), poly(alkylcarbonate), poly(orthoester), polyester, poly(hydroxyvaleric acid), polydioxanone, poly(ethylene, terephtalate), poly(maleic acid), poly(tartaric acid), polyanhydride, polyphosphohazene, poly(amino acids), and all of the copolymers and any mixtures thereof.

In another exemplary embodiment, the material can be based on inorganic composites or organic composites or hybrid inorganic/organic composites. Other, non-degradable materials may be added to the biodegradable implant material, if desired.

Exemplary Openings and Reservoir Function

In the exemplary embodiments of the present invention, the size of the compartment or reservoir of the implant can be modified as desired. For example, the distance between the walls enclosing the lumen or respectively the cross-sectional diameter of the inner compartment of a single strut can be varied. According to an exemplary embodiment of the present invention, the distance or diameters can be in a range of about 100 nm up to 30 centimeter (cm). For expandable devices or stents, it is possible to have a distance or diameter in the range of about 100 nanometer (nm) up to 10 millimeter (mm), such as from about 200 nm up to 500 micrometer (μm), or from about 500 nm to 200 μm. However, these are exemplary embodiments and not limiting values. Without being bound to a specific theory, it may be preferred to determine the ratio between the thickness of a strut or node and to adjust the compartment wall distance or diameter to about 20% up to 95% of the overall thickness of the strut or node (outer dimensions).

In one exemplary embodiment of the present invention, the size of the inner compartment or respective reservoir can be different at different sections of the implant. This can be beneficial particularly, if the release of the beneficial agent shall be modified in terms of available amounts at different locations of the implant. Furthermore, the location, size and number of porous wall sections or additional openings can be used for controlling the release of a beneficial agent, or the absorptive properties of the implant. A higher porosity, larger average pore sizes, or a higher amount of openings with an equidistant distribution may result in a homogeneous release of the beneficial agents. The location of porous wall sections or the openings would be on the outer surface or inner surface, and also laterally at the struts or nodes or combined on all sides. Additionally, the release profile can be accurately controlled by the pore sizes, the porosity, the dimensions of the porous wall or by the size of the openings. Larger sizes can increase the surface/volume ratio and therefore typically result in a higher elution rate of beneficial agents.

In an exemplary embodiment, the sizes of the openings may be between about 5 nm and 400 μm, such as from about 20 nm to 100 μm, and or from about 250 nm to 50 μm. In other exemplary embodiments, the sizes may be varied, so that any combination of different opening sizes—independent of the location and orientation of the openings—can be realized. In specific exemplary embodiments, different sizes on the outer and inner surfaces may be used, particularly, to realize a faster or slower release or absorption rate on the different surfaces, e.g., a fast or slow release at the abluminal side of a target vessel compared to a slower/faster release on the luminal side.

Incorporation of beneficial agents may be carried out by any suitable mean, preferably by dip-coating, spray coating or the like. The beneficial agent may be provided in an appropriate solvent, optionally using additives. The loading of these agents may be carried out under atmospheric, sub-atmospheric pressure or under vacuum. Alternatively, the exemplary loading may be carried out under high pressure. Incorporation of the beneficial agent may be carried out by applying electrical charge to the implant or exposing at least a portion of the implant to a gaseous material including the gaseous or vapor phase of the solvent in which an agent is dissolved or other gases that have a high degree of solubility in the loading solvent. In preferable embodiments, the beneficial agents are provided using carriers that are incorporated into the compartment of the implant. Carriers can be selected from any suitable group of polymers or solvents to select carriers from pH-sensitive polymers, like, for example, however not exclusively: poly(acrylic acid) and derivatives, for example: homopolymers like poly(amino carboxylic acid), poly(acrylic acid), poly(methyl acrylic acid) and their copolymers. This applies likewise for polysaccharides like celluloseacetatephthalate, hydroxylpropylmethylcellulosephthalate, hydroxypropylmethylcellulosesuccinate, celluloseacetatetrimellitate and chitosan.

In certain exemplary embodiments, it can be preferable to select carriers from temperature sensitive polymers, like for example, however not exclusively: poly(N-isopropylacrylamide-co-sodium-acrylate-co-n-N-alkylacrylamide), poly(N-methyl-N-n-propylacrylamide), poly(N-methyl-N-isopropylacrylamide), poly(N—N-propylmethacrylamide), poly(N-isopropylacrylamide), poly(N,N-diethylacrylamide), poly(N-isopropylmethacrylamide), poly(N-cyclopropylacrylamide), poly(N-ethylacrylamide), poly(N-ethylmethylacrylamide), poly(N-methyl-N-ethylacrylamide), poly(N-cyclopropylacrylamide). Other polymers suitable to be used as a carrier with thermogel characteristics are hydroxypropylcellulose, methylcellulose, hydroxypropylmethylcellulose, ethylhydroxyethylcellulose and pluronics like F-127, L-122, L-92, L-81, L-61. Preferable carrier polymers include also, however not exclusively, functionalized styrene, like amino styrene, functionalized dextrane and polyamino acids. Furthermore polyamino acids, (poly-D-amino acids as well as poly-L-amino acids), for example polylysine, and polymers which contain lysine or other suitable amino acids. Other useful polyamino acids are polyglutamic acids, polyaspartic acid, copolymers of lysine and glutamine or aspartic acid, copolymers of lysine with alanine, tyrosine, phenylalanine, serine, tryptophan and/or proline.

In exemplary embodiments of devices, the porous reservoir function may also be determined by the thickness of the walls of the porous compartment and the elastomechanical properties of the implant material. Without wishing to be bound to a specific theory, the decrease of thickness, or respectively increase of pore sizes and/or porosity, with a given metal material for example will result in an increase of plastic deformation of the wall. Expansion or compression of the implant then causes a deformation of the wall and—depending on the extent of elastic and/or plastic deformation—an irreversible or reversible compression of the reservoir. This function can be tailored by a person skilled in the art, for example by using finite element analysis or validating the implant in practice. The increase in pressure with the compartment or reservoir then results in a temporary or repetitive increase of elution of incorporated beneficial agents. This function can be tailored toward a single or multiple bolus elutions, if preferable. Using organic materials with particularly elastic properties, like selecting an elastomer material, can also result in a functional implant that releases bolus-like any beneficial agent upon physiologic increases of pressure with the living body.

Exemplary beneficial agents can be incorporated partially or completely into the compartment or reservoir of the implant. Furthermore, it is also one aspect of the present invention to optionally coat the exemplary implant with beneficial agents partially or completely.

Biologically, therapeutically or pharmaceutically active agents according to the present invention may be a drug, pro-drug or even a targeting group or a drug comprising a targeting group. The active agents may be in crystalline, polymorphous or amorphous form or any combination thereof in order to be used in the present invention.

The active ingredients may be in crystalline, polymorphous or amorphous form or any combination thereof in order to be used in the present invention.

Suitable therapeutically active agents may be selected from the group of enzyme inhibitors, hormones, cytokines, growth factors, receptor ligands, antibodies, antigens, ion binding agents, such as crown ethers and chelating compounds, substantial complementary nucleic acids, nucleic acid binding proteins including transcriptions factors, toxins etc. Examples of such active agents are, for example, cytokines, such as erythropoietine (EPO), thrombopoietine (TPO), interleukines (including IL-I to IL-17), insulin, insulin-like growth factors (including IGF-1 and IGF-2), epidermal growth factor (EGF), transforming growth factors (including TGF-alpha and TGF-beta), human growth hormone, transferrine, low density lipoproteins, high density lipoproteins, leptine, VEGF, PDGF, ciliary neurotrophic factor, prolactine, adrenocorticotropic hormone (ACTH), calcitonin, human chorionic gonadotropin, cortisol, estradiol, follicle stimulating hormone (FSH), thyroid-stimulating hormone (TSH), leutinizing hormone (LH), progesterone, testosterone, toxins including ricine and further active agents, such as those included in Physician's Desk Reference, 58th Edition, Medical Economics Data Production Company, Montvale, N.J., 2004 and the Merck Index, 13th Edition (particularly pages Ther-1 to Ther-29).

In an exemplary embodiment, the therapeutically active agent can be selected from the group of drugs for the therapy of oncological diseases and cellular or tissue alterations. Suitable therapeutic agents are, e.g., antineoplastic agents, including alkylating agents, such as alkyl sulfonates, e.g., busulfan, improsulfan, piposulfane, aziridines, such as benzodepa, carboquone, meturedepa, uredepa; ethyleneimine and methylmelamines, such as altretamine, triethylene melamine, triethylene phosphoramide, triethylene thiophosphoramide, trimethylolmelamine; so-called nitrogen mustards, such as chlorambucil, chlornaphazine, cyclophosphamide, estramustine, ifosfamide, mechlorethamine, mechlorethaminoxide hydrochloride, melphalan, novembichin, phenesterine, prednimustine, trofosfamide, uracil mustard; nitroso urea-compounds, such as carmustine, chlorozotocin, fotenmustine, lomustine, nimustine, ranimustine; dacarbazine, mannomustine, mitobranitol, mitolactol; pipobroman; doxorubicin and cis-platinum and its derivatives, etc., combinations and/or derivatives of any of the foregoing.

In a further exemplary embodiment, the therapeutically active agent can be selected from the group of anti-viral and anti-bacterial agents, such as aclacinomycin, actinomycin, anthramycin, azaserine, bleomycin, cuctinomycin, carubicin, carzinophilin, chromomycines, ductinomycin, daunorubicin, 6-diazo-5-oxn-1-norieucin, doxorubicin, epirubicin, mitomycins, mycophenolsäure, mogalumycin, olivomycin, peplomycin, plicamycin, porfiromycin, puromycin, streptonigrin, streptozocin, tubercidin, ubenimex, zinostatin, zorubicin, aminoglycosides or polyenes or macrolid-antibiotics, etc., combinations and/or derivatives of any of the foregoing.

In a further exemplary embodiment, the therapeutically active agent may include a radio-sensitizer drug, or a steroidal or non-steroidal anti-inflammatory drug.

In a further exemplary embodiment, the therapeutically active agent can be selected from agents referring to angiogenesis, such as e.g. endostatin, angiostatin, interferones, platelet factor 4 (PF4), thrombospondin, transforming growth factor beta, tissue inhibitors of the metalloproteinases-1, -2 and -3 (TIMP-1, -2 and -3), TNP-470, marimastat, neovastat, BMS-275291, COL-3, AG3340, thalidomide, squalamine, combrestastatin, SU5416, SU6668, IFN-[alpha], EMD121974, CAI, IL-12 and IM862 etc., combinations and/or derivatives of any of the foregoing.

In a further exemplary embodiment, the therapeutically-active agent may be selected from the group of nucleic acids, wherein the term nucleic acids also comprises oligonucleotides wherein at least two nucleotides are covalently linked to each other, for example in order to provide gene therapeutic or antisense effects. Nucleic acids preferably comprise phosphodiester bonds, which also comprise those which are analogues having different backbones. Analogues may also contain backbones, such as, for example, phosphoramide (Beaucage et al., Tetrahedron 49(10):1925 (1993) and the references cited therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)); phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidit-compounds (see Eckstein, Oligonucleotides and Analogues: A Practical Approach, Oxford University Press), and peptide-nucleic acid-backbones and their compounds (see Egholm, J. Am. Chem. Soc. 114:1895 (1992); Meier et al., Chem. Int. Ed. Engl: 31:1008 (1992); Nielsen, Nature, 365:566 (1993); Carlsson et al., Nature 380:207 (1996), wherein these references are incorporated by reference herein. further analogues are those having ionic backbones, see Denpcy et al., Proc. Natl. Acad. Sci. USA 92:6097 (1995), or non-ionic backbones, see U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996), and non-ribose-backbones, including those which are described in U.S. Pat. Nos. 5,235,033 and 5,034,506, and in chapters 6 and 7 of ASC Symposium Series 580, “Carbohydrate Modifications in Antisense Research”, Ed. Y. S. Sanghui and P. Dan Cook. The nucleic acids having one or more carbocylic sugars are also suitable as nucleic acids for use in the present invention, see Jenkins et al., Chemical Society Review (1995), pages 169 to 176 as well as others which are described in Rawls, C & E News, 2 Jun. 1997, page 36. Besides the selection of the nucleic acids and nucleic acid analogues known in the prior art, also a mixture of naturally occurring nucleic acids and nucleic acid analogues or mixtures of nucleic acid analogues may be used.

In a further exemplary embodiment, the therapeutically active agent is selected from the group of metal ion complexes, as described in International Applications PCT/US95/16377, PCT/US95/16377, PCT/US96/19900 and PCT/US96/15527, whereas such agents reduce or inactivate the bioactivity of their target molecules, preferably proteins, such as enzymes.

Therapeutically active agents may also include anti-migratory, anti-proliferative or immune-suppressive, anti-inflammatory or re-endotheliating agents, such as, e.g., everolimus, tacrolimus, sirolimus, mycofenolate-mofetil, rapamycin, paclitaxel, actinomycine D, angiopeptin, batimastate, estradiol, statines and others, their derivatives and analogues.

Active agents or combinations of active agents may further be selected from heparin, synthetic heparin analogs (e.g., fondaparinux), hirudin, antithrombin III, drotrecogin alpha; fibrinolytics, such as alteplase, plasmin, lysokinases, factor XIIa, prourokinase, urokinase, anistreplase, streptokinase; platelet aggregation inhibitors, such as acetylsalicylic acid [aspirin], ticlopidine, clopidogrel, abciximab, dextrans; corticosteroids, such as alclometasone, amcinonide, augmented betamethasone, beclomethasone, betamethasone, budesonide, cortisone, clobetasol, clocortolone, desonide, desoximetasone, dexamethasone, fluocinolone, fluocinonide, flurandrenolide, flunisolide, fluticasone, halcinonide, halobetasol, hydrocortisone, methylprednisolone, mometasone, prednicarbate, prednisone, prednisolone, triamcinolone; so-called non-steroidal anti-inflammatory drugs (NSAIDs), such as diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, celecoxib, rofecoxib; cytostatics, such as alkaloides and podophyllum toxins, such as vinblastine, vincristine; alkylating agents, such as nitrosoureas, nitrogen lost analogs; cytotoxic antibiotics, such as daunorubicin, doxorubicin and other anthracyclines and related substances, bleomycin, mitomycin; antimetabolites, such as folic acid analogs, purine analogs or pyrimidine analogs; paclitaxel, docetaxel, sirolimus; platinum compounds, such as carboplatin, cisplatin or oxaliplatin; amsacrin, irinotecan, imatinib, topotecan, interferon-alpha 2a, interferon-alpha 2b, hydroxycarbamide, miltefosine, pentostatin, porfimer, aldesleukin, bexaroten, tretinoin; antiandrogens and antiestrogens; antiarrythmics in particular class I antiarrhythmic, such as antiarrhythmics of the quinidine type, quinidine, dysopyramide, ajmaline, prajmalium bitartrate, detajmium bitartrate; antiarrhythmics of the lidocaine type, e.g., lidocaine, mexiletin, phenyloin, tocainid; class Ic antiarrhythmics, e.g., propafenon, flecainid (acetate); class II antiarrhythmics beta-receptor blockers, such as metoprolol, esmolol, propranolol, metoprolol, atenolol, oxprenolol; class III antiarrhythmics, such as amiodarone, sotalol; class IV antiarrhythmics, such as diltiazem, verapamil, gallopamil; other antiarrhythmics, such as adenosine, orciprenaline, ipratropium bromide; agents for stimulating angiogenesis in the myocardium, such as vascular endothelial growth factor (VEGF), basic fibroblast growth factor (bFGF), non-viral DNA, viral DNA, endothelial growth factors: FGF-1, FGF-2, VEGF, TGF; antibiotics, monoclonal antibodies, anticalins; stem cells, endothelial progenitor cells (EPC); digitalis glycosides, such as acetyl digoxin/metildigoxin, digitoxin, digoxin; cardiac glycosides, such as ouabain, proscillaridin; antihypertensives, such as CNS active antiadrenergic substances, e.g., methyldopa, imidazoline receptor agonists; calcium channel blockers of the dihydropyridine type, such as nifedipine, nitrendipine; ACE inhibitors: quinaprilate, cilazapril, moexipril, trandolapril, spirapril, imidapril, trandolapril; angiotensin II antagonists: candesartancilexetil, valsartan, telmisartan, olmesartanmedoxomil, eprosartan; peripherally active alpha-receptor blockers, such as prazosin, urapidil, doxazosin, bunazosin, terazosin, indoramin; vasodilatators, such as dihydralazine, diisopropylamine dichloracetate, minoxidil, nitroprusside sodium; other antihypertensives, such as indapamide, co-dergocrine mesylate, dihydroergotoxin methanessulfonate, cicletanin, bosentan, fludrocortisone; phosphodiesterase inhibitors, such as milrinon, enoximon and antihypotensives, such as in particular adrenergic and dopaminergic substances, such as dobutamine, epinephrine, etilefrine, norfenefrine, norepinephrine, oxilofrine, dopamine, midodrine, pholedrine, ameziniummetil; and partial adrenoceptor agonists, such as dihydroergotamine; fibronectin, polylysine, ethylene vinyl acetate, inflammatory cytokines, such as: TGF, PDGF, VEGF, bFGF, TNF, NGF, GM-CSF, IGF-a, IL-1, IL 8, IL-6, growth hormone; as well as adhesive substances, such as cyanoacrylates, beryllium, silica; and growth factors, such as erythropoetin, hormones, such as corticotropins, gonadotropins, somatropins, thyrotrophins, desmopressin, terlipressin, pxytocin, cetrorelix, corticorelin, leuprorelin, triptorelin, gonadorelin, ganirelix, buserelin, nafarelin, goserelin, as well as regulatory peptides, such as somatostatin, octreotid; bone and cartilage stimulating peptides, bone morphogenetic proteins (BMPs), in particulary recombinant BMPs, such as recombinant human BMP-2 (rhBMP-2), bisphosphonate (e.g., risedronate, pamidronate, ibandronate, zoledronic acid, clodronsaure, etidronsaure, alendronic acid, tiludronic acid), fluorides, such as disodium fluorophosphate, sodium fluoride; calcitonin, dihydrotachystyrol; growth factors and cytokines, such as epidermal growth factor (EGF), platelet-derived growth factor (PDGF), fibroblast growth factors (FGFs), transforming growth factors-b (TGFs-b), transforming growth factor-a (TGF-a), erythropoietin (EPO), insulin-like growth factor-I (IGF-I), insulin-like growth factor-II (IGF-II), interleukin-1 (IL-1), interleukin-2 (IL-2), interleukin-6 (IL-6), interleukin-8 (IL-8), tumor necrosis factor-a (TNF-a), tumor necrosis factor-b (TNF-b), interferon-g (INF-g), colony stimulating factors (CSFs); monocyte chemotactic protein, fibroblast stimulating factor 1, histamine, fibrin or fibrinogen, endothelin-1, angiotensin II, collagens, bromocriptine, methysergide, methotrexate, carbon tetrachloride, thioacetamide and ethanol; as well as silver (ions), titanium dioxide, antibiotics and anti-infective drugs, such as in particular β-lactam antibiotics, e.g., β-lactamase-sensitive penicillins, such as benzyl penicillins (penicillin G), phenoxymethylpenicillin (penicillin V); β-lactamase-resistant penicillins, such as aminopenicillins, e.g., amoxicillin, ampicillin, bacampicillin; acylaminopenicillins, such as mezlocillin, piperacillin; carboxypenicillins, cephalosporins, such as cefazoline, cefuroxim, cefoxitin, cefotiam, cefaclor, cefadroxil, cefalexin, loracarbef, cefixim, cefuroximaxetil, ceftibuten, cefpodoximproxetil, cefpodoximproxetil; aztreonam, ertapenem, meropenem; β-lactamase inhibitors, such as sulbactam, sultamicillintosylate; tetracyclines, such as doxycycline, minocycline, tetracycline, chlorotetracycline, oxytetracycline; aminoglycosides, such as gentamicin, neomycin, streptomycin, tobramycin, amikacin, netilmicin, paromomycin, framycetin, spectinomycin; macrolide antibiotics, such as azithromycin, clarithromycin, erythromycin, roxithromycin, spiramycin, josamycin; lincosamides, such as clindamycin, lincomycin; gyrase inhibitors, such as fluoroquinolones, e.g., ciprofloxacin, ofloxacin, moxifloxacin, norfloxacin, gatifloxacin, enoxacin, fleroxacin, levofloxacin; quinolones, such as pipemidic acid; sulfonamides, trimethoprim, sulfadiazine, sulfalene; glycopeptide antibiotics, such as vancomycin, teicoplanin; polypeptide antibiotics, such as polymyxins, e.g., colistin, polymyxin-b, nitroimidazole derivates, e.g., metronidazole, tinidazole; aminoquinolones, such as chloroquin, mefloquin, hydroxychloroquin; biguanids, such as proguanil; quinine alkaloids and diaminopyrimidines, such as pyrimethamine; amphenicols, such as chloramphenicol; rifabutin, dapson, fusidic acid, fosfomycin, nifuratel, telithromycin, fusafungin, fosfomycin, pentamidine diisethionate, rifampicin, taurolidin, atovaquon, linezolid; virus static, such as aciclovir, ganciclovir, famciclovir, foscarnet, inosine-(dimepranol-4-acetamidobenzoate), valganciclovir, valaciclovir, cidofovir, brivudin; antiretroviral active ingredients (nucleoside analog reverse-transcriptase inhibitors and derivatives), such as lamivudine, zalcitabine, didanosine, zidovudin, tenofovir, stavudin, abacavir; non-nucleoside analog reverse-transcriptase inhibitors: amprenavir, indinavir, saquinavir, lopinavir, ritonavir, nelfinavir; amantadine, ribavirine, zanamivir, oseltamivir or lamivudine, as well as any combinations and mixtures thereof.

In an alternative exemplary embodiment of the present invention, the active agents can be encapsulated in polymers, vesicles, liposomes or micelles.

Suitable diagnostically active agents for use in an exemplary embodiment of the present invention can be, e.g., signal generating agents or materials, which may be used as markers. Such signal generating agents include materials which in physical, chemical and/or biological measurement and verification methods lead to detectable signals, for example in image-producing methods. It is not important for the present invention, whether the signal processing is carried out exclusively for diagnostic or therapeutic purposes. Typical imaging methods are for example radiographic methods, which are based on ionizing radiation, for example conventional X-ray methods and X-ray based split image methods, such as computer tomography, neutron transmission tomography, radiofrequency magnetization, such as magnetic resonance tomography, further by radionuclide-based methods, such as scintigraphy, Single Photon Emission Computed Tomography (SPECT), Positron Emission Computed Tomography (PET), ultrasound-based methods or fluoroscopic methods or luminescence or fluorescence based methods, such as Intravasal Fluorescence Spectroscopy, Raman spectroscopy, Fluorescence Emission Spectroscopy, Electrical Impedance Spectroscopy, colorimetry, optical coherence tomography, etc, further Electron Spin Resonance (ESR), Radio Frequency (RF) and Microwave Laser and similar methods.

Signal generating agents can be metal-based from the group of metals, metal oxides, metal carbides, metal nitrides, metal oxynitrides, metal carbonitrides, metal oxycarbides, metal oxynitrides, metal oxycarbonitrides, metal hydrides, metal alkoxides, metal halides, inorganic or organic metal salts, metal polymers, metallocenes, and other organometallic compounds.

Preferable metal-based agents are e.g. nanomorphous nanoparticles from metals, metal oxides semiconductors as defined above as the metal-based particles, or mixtures thereof. In this regard, it may be preferable to select at least a part of the metal-based particles from those materials capable of functioning as signal generating agents, for example to mark the implant for better visibility and localization in the body after implantation.

Further, signal producing metal-based agents can be selected from salts or metal ions, which preferably have paramagnetic properties, for example lead (II), bismuth (II), bismuth (III), chromium (III), manganese (II), manganese (III), iron (II), iron (III), cobalt (II), nickel (II), copper (II), praseodymium (III), neodymium (III), samarium (III), or ytterbium (III), holmium (III) or erbium (III) etc. Based on especially pronounced magnetic moments, especially gadolinium (III), terbium (III), dysprosium (III), holmium (III) and erbium (III) are mostly preferable. Further one can select from radioisotopes. Examples of a few applicable radioisotopes include H 3, Be 10, O 15, Ca 49, Fe 60, In 111, Pb 210, Ra 220, Ra 224 and the like. Typically such ions are present as chelates or complexes, wherein for example as chelating agents or ligands for lanthanides and paramagnetic ions compounds, such as diethylenetriamine pentaacetic acid (“DTPA”), ethylenediamine tetra acetic acid (“EDTA”), or tetraazacyclododecane-N,N′,N″,N′″-tetra acetic acid (“DOTA”) are used. Other typical organic complexing agents are for example published in Alexander, Chem. Rev. 95:273-342 (1995) and Jackels, Pharm. Med. Imag, Section III, Chap. 20, p 645 (1990). Other usable chelating agents may be found in U.S. Pat. Nos. 5,155,215; 5,087,440; 5,219,553; 5,188,816; 4,885,363; 5,358,704; 5,262,532, and Meyer et al., Invest. Radiol. 25: S53 (1990), further U.S. Pat. Nos. 5,188,816, 5,358,704, 4,885,363, and 5,219,553. In addition, salts and chelates from the lanthanide group with the atomic numbers 57-83 or the transition metals with the atomic numbers 21-29, or 42 or 44 may be incorporated into the implants of exemplary embodiments of the present invention.

In addition suitable can be paramagnetic perfluoroalkyl containing compounds which for example are described in German Patent Application Nos. 196 03 033 and 197 29 013 and International Patent Publication WO 97/26017, further diamagnetic perfluoroalkyl containing substances of the general formula:

R<PF>-L<II>-G<III>,

whereas R<PF> represents a perfluoroalkyl group with 4 to 30 carbon atoms, L<II> stands for a linker and G<III> for a hydrophilic group. The linker L is a direct bond, an —SO2-group or a straight or branched carbon chain with up to 20 carbon atoms which can be substituted with one or more —OH, —COO<->, —SO3-groups and/or if necessary one or more —O—, —S—, —CO—, —CONH—, —NHCO—, —CONR—, —NRCO—, —SO2—, —PO4—, —NH—, —NR-groups, an aryl ring or contain a piperazine, wherein R stands for a C1 to C20 alkyl group, which again can contain and/or have one or a plurality of O atoms and/or be substituted with —COO<-> or SO3-groups.

The hydrophilic group G<III> can be selected from a mono or disaccharide, one or a plurality of —COO<-> or —SO3<->-groups, a dicarboxylic acid, an isophthalic acid, a picolinic acid, a benzenesulfonic acid, a tetrahydropyranedicarboxylic acid, a 2,6-pyridinedicarboxylic acid, a quaternary ammonium ion, an aminopolycarboxcyclic acid, an aminodipolyethyleneglycol sulfonic acid, an aminopolyethyleneglycol group, an SO2-(CH2)2-OH-group, a polyhydroxyalkyl chain with at least two hydroxyl groups or one or a plurality of polyethylene glycol chains having at least two glycol units, wherein the polyethylene glycol chains are terminated by an —OH or —OCH3-group, or similar linkages.

In exemplary embodiments, paramagnetic metals in the form of metal complexes with phthalocyanines may be used to functionalize the implant, especially as described in Phthalocyanine Properties and Applications, Vol. 14, C. C. Leznoff and A. B. P. Lever, VCH Ed. Examples are octa(1,4,7,10-tetraoxaundecyl)Gd-phthalocyanine, octa(1,4,7,10-tetraoxaundecyl)Gd-phthalocyanine, octa(1,4,7,10-tetraoxaundecyl)Mn-phthalocyanine, octa(1,4,7,10-tetraoxaundecyl)Mn-phthalocyanine, as described in U.S. Patent Publication No. 2004/214810.

Super-paramagnetic, ferromagnetic or ferrimagnetic signal generating agents may also be used. For example among magnetic metals, alloys are preferable, among ferrites, such as gamma iron oxide, magnetites or cobalt-, nickel- or manganese-ferrites, corresponding agents are preferably selected, especially particles as described in International Patent Publications WO83/03920, WO83/01738, WO85/02772 and WO89/03675, in U.S. Pat. Nos. 4,452,773 and 4,675,173, in International Patent Publication WO88/00060 as well as U.S. Pat. No. 4,770,183, in International Patent Publication WO90/01295 and in International Patent Publication WO90/01899.

Further, magnetic, paramagnetic, diamagnetic or super paramagnetic metal oxide crystals having diameters of less than about 4000 Angstroms are especially preferable as degradable non-organic diagnostic agents. Suitable metal oxides can be selected from iron oxide, cobalt oxides, iridium oxides or the like, which provide suitable signal producing properties and which have especially biocompatible properties or are biodegradable. Crystalline agents of this group having diameters smaller than 500 Angstroms may be used. These crystals can be associated covalently or non-covalently with macromolecular species. Further, zeolite containing paramagnets and gadolinium containing nanoparticles can be selected from polyoxometallates, preferably of the lanthanides, (e.g., K9GdW10O36).

For optimizing the image producing properties the average particle size of the magnetic signal producing agents may be limited to 5 μm at maximum, such as from about 2 nm up to 1 μm, e.g. from about 5 nm to 200 nm. The super paramagnetic signal producing agents can be chosen for example from the group of so-called SPIOs (super paramagnetic iron oxides) with a particle size larger than about 50 nm or from the group of the USPIOs (ultra small super paramagnetic iron oxides) with particle sizes smaller than 50 nm.

Signal generating agents for imparting further functionality to the implants of embodiments of the present invention can further be selected from endohedral fullerenes, as disclosed for example in U.S. Pat. No. 5,688,486 or International Patent Publication WO 93/15768, or from fullerene derivatives and their metal complexes, such as fullerene species, which comprise carbon clusters having 60, 70, 76, 78, 82, 84, 90, 96 or more carbon atoms. An overview of such species can be gathered from European patent application No. 1331226A2. Metal fullerenes or endohedral carbon-carbon nanoparticles with arbitrary metal-based components can also be selected. Such endohedral fullerenes or endometallo fullerenes may contain for example rare earths, such as cerium, neodymium, samarium, europium, gadolinium, terbium, dysprosium or holmium. The choice of nanomorphous carbon species is not limited to fullerenes, other nanomorphous carbon species, such as nanotubes, onions, etc. may also be applicable.

In another exemplary embodiment fullerene species may be selected from non-endohedral or endohedral forms which contain halogenated, preferably iodated, groups, as described in U.S. Pat. No. 6,660,248.

Generally, mixtures of such signal generating agents of different specifications can also used, depending on the desired properties of the signal generating material properties. The signal producing agents used can have a size of about 0.5 nm to 1,000 nm, preferably about 0.5 nm to 900 nm, especially preferable from about 0.7 to 100 nm, and the may partly replace the metal-based particles. Nanoparticles are easily modifiable based on their large surface to volume ratios. The nanoparticles can for example be modified non-covalently by means of hydrophobic ligands, for example with trioctylphosphine, or be covalently modified. Examples of covalent ligands are thiol fatty acids, amino fatty acids, fatty acid alcohols, fatty acids, fatty acid ester groups or mixtures thereof, for example oleic cid and oleylamine.

In exemplary embodiments of the present invention, the active ingredients, such as signal producing agents can be encapsulated in micelles or liposomes with the use of amphiphilic components, or may be encapsulated in polymeric shells, wherein the micelles/liposomes can have a diameter of about t2 nm to 800 nm, preferably from about 5 to 200 nm, especially preferable from about 10 to 25 nm. The micelles/liposomes may be added to the suspension before molding, to be incorporated into the implant. The size of the micelles/liposomes is, without committing to a specific theory, dependant on the number of hydrophobic and hydrophilic groups, the molecular weight of the nanoparticles and the aggregation number. In aqueous solutions the use of branched or unbranched amphiphilic substances, is especially preferable in order to achieve the encapsulation of signal generating agents in liposomes/micelles. The hydrophobic nucleus of the micelles hereby contains in a exemplary embodiment a multiplicity of hydrophobic groups, preferably between 1 and 200, especially preferable between about 1 and 100 and mostly preferable between about 1 and 30 according to the desired setting of the micelle size.

Hydrophobic groups can consist preferably of hydrocarbon groups or residues or silicon-containing residues, for example polysiloxane chains. Furthermore, they can be selected from hydrocarbon-based monomers, oligomers and polymers, or from lipids or phospholipids or comprise combinations hereof, especially glyceryl esters, such as phosphatidyl ethanolamine, phosphatidyl choline, or polyglycolides, polylactides, polymethacrylate, polyvinylbutylether, polystyrene, polycyclopentadienylmethylnorbornene, polyethylenepropylene, polyethylene, polyisobutylene, polysiloxane. Further for encapsulation in micelles hydrophilic polymers are also selected, especially preferred polystyrenesulfonic acid, poly-N-alkylvinylpyridiniumhalides, poly(meth)acrylic acid, polyamino acids, poly-N-vinylpyrrolidone, polyhydroxyethylmethacrylate, polyvinyl ether, polyethylene glycol, polypropylene oxide, polysaccharides like agarose, dextrane, starches, cellulose, amylose, amylopectin, or polyethylene glycol or polyethylene imine of any desired molecular weight, depending on the desired micelles property. Further, mixtures of hydrophobic or hydrophilic polymers can be used or such lipid-polymer compositions employed. In a further special embodiment, the polymers are used as conjugated block polymers, whereas hydrophobic and also hydrophilic polymers or any desired mixtures there of can be selected as 2-, 3- or multi-block copolymers.

Such signal generating agents encapsulated in micelles and incorporated into the porous implant can moreover be functionalized, while linker (groups) are attached at any desired position, preferably amino-, thiol, carboxyl-, hydroxyl-, succinimidyl, maleimidyl, biotin, aldehyde- or nitrilotriacetate groups, to which any desired corresponding chemically covalent or non-covalent other molecules or compositions can be bound according to the prior art. Here, especially biological molecules, such as proteins, peptides, amino acids, polypeptides, lipoproteins, glycosaminoglycanes, DNA, RNA or similar biomolecules are preferable especially.

Signal generating agents may also be selected from non-metal-based signal generating agents, for example from the group of X-ray contrast agents, which can be ionic or non-ionic. Among the ionic contrast agents are included salts of 3-acetyl amino-2,4-6-triiodobenzoic acid, 3,5-diacetamido-2,4,6-triiodobenzoic acid, 2,4,6-triiodo-3,5-dipropionamido-benzoic acid, 3-acetyl amino-5-((acetyl amino)methyl)-2,4,6-triiodobenzoic acid, 3-acetyl amino-5-(acetyl methyl amino)-2,4,6-triiodobenzoic acid, 5-acetamido-2,4,6-triiodo-N-((methylcarbamoyl)methyl)-isophthalamic acid, 5-(2-methoxyacetamido)-2,4,6-triiodo-N-[2-hydroxy-1-(methylcarbamoyl)-ethoxy 1]-isophthalamic acid, 5-acetamido-2,4,6-triiodo-N-methylisophthalamic acid, 5-acetamido-2,4,6-triiodo-N-(2-hydroxyethyl)-isophthalamic acid 2-[[2,4,6-triiodo-3-[(1-oxobutyl)-amino]phenyl]methyl]-butanoic acid, beta-(3-amino-2,4,6-triiodophenyl)-alpha-ethyl-propanoic acid, 3-ethyl-3-hydroxy-2,4,6-triiodophenyl-propanoic acid, 3-[[(dimethylamino)-methyl]amino]-2,4,6-triiodophenyl-propanoic acid (see Chem. Ber. 93: 2347 (1960)), alpha-ethyl-(2,4,6-triiodo-3-(2-oxo-1-pyrrolidinyl)-phenyl)-propanoic acid, 2-[2-[3-(acetyl amino)-2,4,6-triiodophenoxy]ethoxymethyl]butanoic acid, N-(3-amino-2,4,6-triiodobenzoyl)-N-phenyl-.beta.-aminopropanoic acid, 3-acetyl-[(3-amino-2,4,6-triiodophenyl)amino]-2-methylpropanoic acid, 5-[(3-amino-2,4,6-triiodophenyl)methyl amino]-5-oxypentanoic acid, 4-[ethyl-[2,4,6-triiodo-3-(methyl amino)-phenyl]amino]-4-oxo-butanoic acid, 3,3′-oxy-bis[2,1-ethanediyloxy-(1-oxo-2,1-ethanediyl)imino]bis-2,4,6-triiodobenzoic acid, 4,7,10,13-tetraoxahexadecane-1,16-dioyl-bis(3-carboxy-2,4,6-triiodoanilide), 5,5′-(azelaoyldiimino)-bis[2,4,6-triiodo-3-(acetyl amino)methyl-benzoic acid], 5,5′-(apidoldiimino)bis(2,4,6-triiodo-N-methyl-isophthalamic acid), 5,5′-(sebacoyl-diimino)-bis(2,4,6-triiodo-N-methylisophthalamic acid), 5,5-[N,N-diacetyl-(4,9-dioxy-2,11-dihydroxy-1,12-dodecanediyl)diimino]bis(2,4,6-triiodo-N-methyl-isophthalamic acid), 5,5′5″-(nitrilo-triacetyltriimino)tris(2,4,6-triiodo-N-methyl-isophthalamic acid), 4-hydroxy-3,5-diiodo-alpha-phenylbenzenepropanoic acid, 3,5-diiodo-4-oxo-1(4H)-pyridine acetic acid, 1,4-dihydro-3,5-diiodo-1-methyl-4-oxo-2,6-pyridinedicarboxylic acid, 5-iodo-2-oxo-1 (2H)-pyridine acetic acid, and N-(2-hydroxyethyl)-2,4,6-triiodo-5-[2,4,6-triiodo-3-(N-methylacetamido)-5-(methylcarbomoyl)benzamino]acetamido]-isophthalamic acid, and the like, especially preferable, as well as other ionic X-ray contrast agents suggested in the literature, for example in J. Am. Pharm. Assoc., Sci. Ed. 42:721 (1953), Swiss Patent 480071, JACS 78:3210 (1956), German patent 2229360, U.S. Pat. No. 3,476,802, Arch. Pharm. (Weinheim, Germany) 306: 11 834 (1973), J. Med. Chem. 6: 24 (1963), FR-M-6777, Pharmazie 16: 389 (1961), U.S. Pat. Nos. 2,705,726 and 2,895,988, Chem. Ber. 93:2347 (1960), SA-A-68/01614, Acta Radiol. 12: 882 (1972), British Patent No. 870321, Rec. Trav. Chim. 87: 308 (1968), East German Patent No. 67209, German Patent No. 2050217, German Patent 2405652, Farm Ed. Sci. 28: 912 (1973), Farm Ed. Sci. 28: 996 (1973), J. Med. Chem. 9: 964 (1966), Arzheim.-Forsch 14: 451 (1964), SE-A-344166, British Patent No. 1346796, U.S. Pat. Nos. 2,551,696 and 1,993,039, Ann 494: 284 (1932), J. Pharm. Soc. (Japan) 50: 727 (1930), and U.S. Pat. No. 4,005,188.

Examples of applicable non-ionic X-ray contrast agents in accordance with the present invention are metrizamide as described in German DE-A-2031724, iopamidol as described in BE-A-836355, iohexyl as disclosed in British GB-A-1548594, iotrolan as described in European EP-A-33426, iodecimol as described in European EP-A-49745, iodixanol as in EP-A-108638, ioglucol as described in U.S. Pat. No. 4,314,055, ioglucomide as described in BE-A-846657, ioglunioe as in German DE-A-2456685, iogulamide as in BE-A-882309, iomeprol as in European EP-A-26281, iopentol as EP-A-105752, iopromide as in German DE-A-2909439, iosarcol as in German DE-A-3407473, iosimide as in German DE-A-3001292, iotasul as in European EP-A-22056, iovarsul as disclosed in European EP-A-83964 or ioxilan in International Publication WO87/00757.

Agents based on nanoparticle signal generating agents may be selected to impart functionality to the implant, which after release into tissues and cells are incorporated or are enriched in intermediate cell compartments and/or have an especially long residence time in the organism.

Such particles can include water-insoluble agents, a heavy element, such as iodine or barium, PH-50 as monomer, oligomer or polymer (iodinated aroyloxy ester having the empirical formula C19H23I3N2O6, and the chemical names 6-ethoxy-6-oxohexy-3,5-bis (acetyl amino)-2,4,6-triiodobenzoate), an ester of diatrizoic acid, an iodinated aroyloxy ester, or combinations thereof. Particle sizes which can be incorporated by macrophages may be preferable. A corresponding method for this is described in International Publication WO03/039601 and suitable agents are disclosed in the publications U.S. Pat. Nos. 5,322,679, 5,466,440, 5,518,187, 5,580,579, and 5,718,388. Nanoparticles which are marked with signal generating agents or such signal generating agents, such as PH-50, which accumulate in intercellular spaces and can make interstitial as well as extrastitial compartments visible, can be advantageous.

Signal generating agents may also include anionic or cationic lipids, as disclosed in U.S. Pat. No. 6,808,720, for example, anionic lipids, such as phosphatidyl acid, phosphatidyl glycerol and their fatty acid esters, or amides of phosphatidyl ethanolamine, such as anandamide and methanandamide, phosphatidyl serine, phosphatidyl inositol and their fatty acid esters, cardiolipin, phosphatidyl ethylene glycol, acid lysolipids, palmitic acid, stearic acid, arachidonic acid, oleic acid, linoleic acid, linolenic acid, myristic acid, sulfolipids and sulfatides, free fatty acids, both saturated and unsaturated and their negatively charged derivatives, etc. Moreover, halogenated, in particular fluorinated anionic lipids can be preferable in exemplary embodiments. The anionic lipids preferably contain cations from the alkaline earth metals beryllium (Be<+2>), magnesium (Mg<+2>), calcium (Ca<+2>), strontium (Sr<+2>) and barium (Ba<+2>), or amphoteric ions, such as aluminum (Al<+3>), gallium (Ga<+3>), germanium (Ge<+3>), tin (Sn+<4>) or lead (Pb<+2> and Pb<+4>), or transition metals, such as titanium (Ti<+3> and Ti<+4>), vanadium (V<+2> and V<+3>), chromium (Cr<+2> and Cr<+3>), manganese (Mn<+2> and Mn<+3>), iron (Fe<+2> and Fe<+3>), cobalt (Co<+2> and Co<+3>), nickel (Ni<+2> and Ni<+3>), copper (Cu<+2>), zinc (Zn<+2>), zirconium (Zr<+4>), niobium (Nb<+3>), molybdenum (Mo<+2> and Mo<+3>), cadmium (Cd<+2>), indium (In <+3>), tungsten (W<+2> and W<+4>), osmium (Os<+2>, Os<+3> and Os<+4>), iridium (Ir<+2>, Ir<+3> and Ir<+4>), mercury (Hg<+2>) or bismuth (Bi<+3>), and/or rare earths, such as lanthanides, for example lanthanum (La<+3>) and gadolinium (Gd<+3>). Cations can include calcium (Ca<+2>), magnesium (Mg<+2>) and zinc (Zn<+2>) and paramagnetic cations, such as manganese (Mn<+2>) or gadolinium (Gd<+3>).

Cationic lipids may include phosphatidyl ethanolamine, phospatidylcholine, Glycero-3-ethylphosphatidylcholine and their fatty acid esters, di- and tri-methylammoniumpropane, di- and tri-ethylammoniumpropane and their fatty acid esters, and also derivatives, such as N-[1-(2,3-dioleoyloxy)propyl]-N,N,N-trimethylammonium chloride (“DOTMA”); furthermore, synthetic cationic lipids based on for example naturally occurring lipids, such as dimethyldioctadecylammonium bromide, sphingolipids, sphingomyelin, lysolipids, glycolipids, such as for example gangliosides GM1, sulfatides, glycosphingolipids, cholesterol and cholesterol esters or salts, N-succinyldioleoylphosphattidyl ethanolamine, 1,2,-dioleoyl-sn-glycerol, 1,3-dipalmitoyl-2-succinylglycerol, 1,2-dipalmitoyl-sn-3-succinylglycerol, 1-hexadecyl-2-palmitoylglycerophosphatidyl ethanolamine and palmitoyl-homocystein, and fluorinated, derivatized cationic lipids, as disclosed in U.S. Ser. No. 08/391,938. Such lipids are furthermore suitable as components of signal generating liposomes, which especially can have pH-sensitive properties as disclosed in U.S. Patent Publication No. 2004/197392 and incorporated herein explicitly.

Signal generating agents may also include so-called micro bubbles or micro balloons, which contain stable dispersions or suspensions in a liquid carrier substance. Suitable gases may include air, nitrogen, carbon dioxide, hydrogen or noble gases, such as helium, argon, xenon or krypton, or sulfur-containing fluorinated gases, such as sulfur hexafluoride, disulfurdecafluoride or trifluoromethylsulfurpentafluoride, or for example selenium hexafluoride, or halogenated silanes, such as methylsilane or dimethylsilane, further short chain hydrocarbons, such as alkanes, specifically methane, ethane, propane, butane or pentane, or cycloalkanes, such as cyclopropane, cyclobutane or cyclopentane, also alkenes, such as ethylene, propene, propadiene or butene, or also alkynes, such as acetylene or propyne. Further ethers, such as dimethylether may be selected, or ketones, or esters or halogenated short-chain hydrocarbons or any desired mixtures of the above. Examples further include halogenated or fluorinated hydrocarbon gases, such as bromochlorodifluoromethane, chlorodifluoromethane, dichlorodifluoromethan, bromotrifluoromethane, chlorotrifluoromethane, chloropentafluoroethane, dichlorotetrafluoroethane, chlorotrifluoroethylene, fluoroethylene, ethyl fluoride, 1,1-difluoroethane or perfluorohydrocarbons, such as for example perfluoroalkanes, perfluorocycloalkanes, perfluoroalkenes or perfluorinated alkynes. Especially preferable are emulsions of liquid dodecafluoropentane or decafluorobutane and sorbitol, or similar, as disclosed in International Publication WO-A-93/05819.

Preferably such micro bubbles are selected, which are encapsulated in compounds having the structure R1-X-Z; R2-X-Z; or R3-X-Z′ whereas R1, R2 comprises and R3 hydrophobic groups selected from straight chain alkylenes, alkyl ethers, alkyl thiol ethers, alkyl disulfides, polyfluoroalkylenes and polyfluoroalkylethers, Z comprises a polar group from CO2-M<+>, SO3<->M<+>, SO4<-> M<+>, PO3<->M<+>, PO4<->M<+>2, N(R)4<+> or a pyridine or substituted pyridine, and a zwitterionic group, and finally X represents a linker which binds the polar group with the residues.

Gas-filled or in situ out-gassing micro spheres having a size of less than about 1000 μm can be further selected from biocompatible synthetic polymers or copolymers which comprise monomers, dimers or oligomers or other pre-polymer to pre-stages of the following polymerizable substances: acrylic acid, methacrylic acid, ethyleneimine, crotonic acid, acryl amide, ethyl acrylate, methylmethacrylate, 2-hydroxyethylmethacrylate (HEMA), lactonic acid, glycolic acid, [epsilon]caprolactone, acrolein, cyanoacrylate, bisphenol A, epichlorhydrin, hydroxyalkylacrylate, siloxane, dimethylsiloxane, ethylene oxide, ethylene glycol, hydroxyalkylmethacrylate, N-substituted acryl amide, N-substituted methacrylamides, N-vinyl-2-pyrrolidone, 2,4-pentadiene-1-ol, vinyl acetate, acrylonitrile, styrene, p-aminostyrene, p-aminobenzylstyrene, sodium styrenesulfonate, sodium-2-sulfoxyethylmethacrylate, vinyl pyridine, aminoethylmethacrylate, 2-methacryloyloxytrimethylammonium chloride, and polyvinylidenes, such as polyfunctional cross-linkable monomers, such as for example N,N′-methylene-bis-acrylamide, ethylene glycol dimethacrylate, 2,2′-(p-phenylenedioxy)-diethyldimethacrylate, divinylbenzene, triallylamine and methylene-bis-(4-phenyl-isocyanate), including any desired combinations thereof. Preferable polymers contain polyacrylic acid, polyethyleneimine, polymethacrylic acid, polymethylmethacrylate, polysiloxane, polydimethylsiloxane, polylactonic acid, poly([epsilon]-caprolactone), epoxy resins, poly(ethylene oxide), poly(ethylene glycol), and polyamides (e.g. Nylon) and the like, or any arbitrary mixtures thereof. Preferable copolymers contain among others polyvinylidene-polyacrylonitrile, polyvinylidene-polyacrylonitrile-polymethylmethacrylate, and polystyrene-polyacrylonitrile and the like, or any desired mixtures thereof. Methods for manufacture of such micro spheres are published for example in U.S. Pat. Nos. 4,179,546, 3,945,956 and 4,108,806, Japan Kokai Tokkyo Koho 62 286534, British Patent No. 1,044,680, U.S. Pat. Nos. 3,293,114, 3,401,475, 3,479,811, 3,488,714, 3,615,972, 4,549,892, 4,540,629, 4,421,562, 4,420,442, 4,898,734, 4,822,534, 3,732,172, 3,594,326, 3,015,128, Deasy, Microencapsulation and Related Drug Processes, Vol. 20, Chapters. 9 and 10, pp. 195-240 (Marcel Dekker, Inc., N.Y., 1984), Chang et al., Canadian J of Physiology and Pharmacology, Vol 44, pp. 115-129 (1966), and Chang, Science, Vol. 146, pp. 524-525 (1964).

Other signal generating agents can be selected from agents, which are transformed into signal generating agents in organisms using in-vitro or in-vivo cells, cells as a component of cell cultures, of in-vitro tissues, or cells as a component of multicellular organisms, such as, for example, fungi, plants or animals, in exemplary embodiments from mammals, such as mice or humans. Such agents can be made available in the form of vectors for the transfection of multicellular organisms, wherein the vectors contain recombinant nucleic acids for the coding of signal generating agents. In exemplary embodiments this may be done with signal generating agents, such as metal binding proteins. It can be preferable to choose such vectors from the group of viruses for example from adeno viruses, adeno virus associated viruses, herpes simplex viruses, retroviruses, alpha viruses, pox viruses, arena-viruses, vaccinia viruses, influenza viruses, polio viruses or hybrids of any of the above.

Such signal generating agents may be used in combination with delivery systems, e.g. in order to incorporate nucleic acids, which are suitable for coding for signal generating agents, into the target structure. Virus particles for the transfection of mammalian cells may be used, wherein the virus particle contains one or a plurality of coding sequence/s for one or a plurality of signal generating agents as described above. In these cases the particles can be generated from one or a plurality of the following viruses: adeno viruses, adeno virus associated viruses, herpes simplex viruses, retroviruses, alpha viruses, pox viruses, arena-viruses, vaccinia viruses, influenza viruses and polio viruses.

These signal generating agents can be made available from colloidal suspensions or emulsions, which are suitable to transfect cells, preferably mammalian cells, wherein these colloidal suspensions and emulsions contain those nucleic acids which possess one or a plurality of the coding sequence(s) for signal generating agents. Such colloidal suspensions or emulsions can include macromolecular complexes, nano capsules, micro spheres, beads, micelles, oil-in-water- or water-in-oil emulsions, mixed micelles and liposomes or any desired mixture of the above.

In addition, cells, cell cultures, organized cell cultures, tissues, organs of desired species and non-human organisms can be chosen which contain recombinant nucleic acids having coding sequences for signal generating agents. In exemplary embodiments organisms can include mouse, rat, dog, monkey, pig, fruit fly, nematode worms, fish or plants or fungi. Further, cells, cell cultures, organized cell cultures, tissues, organs of desired species and non-human organisms can contain one or a plurality of vectors as described above.

Signal generating agents can be produced in vivo from proteins and made available as described above. Such agents can be directly or indirectly signal producing, while the cells produce (direct) a signal producing protein through transfection, or produce a protein which induces (indirect) the production of a signal producing protein. These signal generating agents are, e.g., detectable in methods, such as MRI while the relaxation times T1, T2, or both are altered and lead to signal producing effects which can be processed sufficiently for imaging. Such proteins can include protein complexes, such as metalloprotein complexes. Direct signal producing proteins can include such metalloprotein complexes which are formed in the cells. Indirect signal producing agents can include proteins or nucleic acids, for example, which regulate the homeostasis of iron metabolism, the expression of endogenous genes for the production of signal generating agents, and/or the activity of endogenous proteins with direct signal generating properties, for example Iron Regulatory Protein (IRP), transferrin receptor (for the take-up of Fe), erythroid-5-aminobevulinate synthase (for the utilization of Fe, H-Ferritin and L-Ferritin for the purpose of Fe storage). In exemplary embodiments both types of signal generating agents, that is direct and indirect, may be combined with each other, for example an indirect signal generating agent, which regulates the iron-homeostasis and a direct agent, which represents a metal binding protein.

In certain exemplary embodiments, where metal-binding polypeptides are selected as indirect agents, it can be advantageous if the polypeptide binds to one or a plurality of metals which possess signal generating properties. Metals with unpaired electrons in the Dorf orbitals may be used, such as for example Fe, Co, Mn, Ni, Gd etc., wherein especially Fe is available in high physiological concentrations in organisms. Such agents may form metal-rich aggregates, for example crystalline aggregates, whose diameters are larger than about 10 picometers, preferably larger than about 100 picometers, 1 nm, 10 nm or even further preferable larger than about 100 nm.

In addition, metal-binding compounds, which have sub-nanomolar affinities with dissociation constants of less than about 10-15 M, 10-2 M or smaller may be used to impart functionality for the implant. Typical polypeptides or metal-binding proteins are lactoferrin, ferritin, or other dimetallocarboxylate proteins, or so-called metal catcher with siderophoric groups, such as hemoglobin. A possible method for preparation of such signal generating agents, their selection and the possible direct or indirect agents which are producible in vivo and are suitable as signal generating agents is described in International Publication WO 03/075747.

Another group of signal generating agents can be photo physically signal producing agents which consist of dyestuff-peptide-conjugates. Such dyestuff-peptide-conjugates can provide a wide spectrum of absorption maxima, for example polymethin dyestuffs, such as cyanine-, merocyanine-, oxonol- and squarilium dyestuffs. From the class of the polymethin dyestuffs the cyanine dyestuffs, e.g. the indole structure based indocarbo-, indodicarbo- and indotricarbocyanines, can be suitable. Such dyestuffs can be substituted with suitable linking agents and can be functionalized with other groups as desired, see also German Patent Application No. 19917713.

The signal generating agents can further be functionalized as desired. The functionalization by means of so-called “Targeting” groups is meant to include functional chemical compounds which link the signal generating agent or its specifically available form (encapsulation, micelles, micro spheres, vectors etc.) to a specific functional location, or to a determined cell type, tissue type or other desired target structures. Targeting groups can permit the accumulation of signal-producing agents in or at specific target structures. Therefore, the targeting groups can be selected from such substances, which are principally suitable to provide a purposeful enrichment of the signal generating agents in their specifically available form by physical, chemical or biological routes or combinations thereof. Useful targeting groups can therefore include antibodies, cell receptor ligands, hormones, lipids, sugars, dextrane, alcohols, bile acids, fatty acids, amino acids, peptides and nucleic acids, which can be chemically or physically attached to signal-generating agents, in order to link the signal-generating agents into/onto a specifically desired structure. Exemplary targeting groups may include those which enrich signal-generating agents in/on a tissue type or on surfaces of cells. Here may not be necessary for the function, that the signal generating agent be taken up into the cytoplasm of the cells. Peptides can be targeting groups, for example chemotactic peptides that are used to visualize inflammation reactions in tissues by means of signal generating agents; see also International Publication WO 97/14443.

Antibodies can be used, including antibody fragments, Fab, Fab2, Single Chain Antibodies (for example Fv), chimerical antibodies, moreover antibody-like substances, for example so-called anticalines, wherein it may not be important whether the antibodies are modified after preparation, recombinants are produced or whether they are human or non-human antibodies. Humanized or human antibodies may be used, such as chimerical immunoglobulines, immunoglobulin chains or fragments (such as Fv, Fab, Fab′, F(ab″)2 or other antigen-binding subsequences of antibodies, which may partly contain sequences of non-human antibodies; humanized antibodies may include human immunoglobulines (receptor or recipient antibody), in which groups of a CDR (Complementary Determining Region) of the receptor are replaced through groups of a CDR of a non-human (spender or donor antibody), wherein the spender species for example, mouse, rabbit or other has appropriate specificity, affinity, and capacity for the binding of target antigens. In a few forms the Fv framework groups of the human immunglobulines are replaced by means of corresponding non-human groups. Humanized antibodies can moreover contain groups which either do not occur in either the CDR or Fv framework sequence of the spender or the recipient. Humanized antibodies essentially comprise substantially at least one or preferably two variable domains, in which all or substantial components of the CDR components of the CDR regions or Fv framework sequences correspond with those of the non-human immunoglobulin, and all or substantial components of the FR regions correspond with a human consensus-sequence. Targeting groups can also include hetero-conjugated antibodies. The functions of the selected antibodies or peptides include cell surface markers or molecules, particularly of cancer cells, wherein here a large number of known surface structures are known, such as HER2, VEGF, CA15-3, CA 549, CA 27.29, CA 19, CA 50, CA242, MCA, CA125, DE-PAN-2, etc.

Moreover, targeting groups may contain the functional binding sites of ligands and which are suitable for binding to any desired cell receptors. Examples of target receptors include receptors of the group of insulin receptors, insulin-like growth factor receptor (e IGF-1 and IGF-2), growth hormone receptor, glucose transporters (particularly GLUT 4 receptor), transferrin receptor (transferrin), Epidermal Growth Factor receptor (EGF), low density lipoprotein receptor, high density lipoprotein receptor, leptin receptor, estrogen receptor; interleukin receptors including IL-1, IL-2, IL-3, IL-4, IL-5, IL-6, IL-7, IL-8, IL-9, IL-11, IL-12, IL-13, IL-15, and IL-17 receptor, VEGF receptor (VEGF), PDGF receptor (PDGF), Transforming Growth Factor receptor (including TGF-[alpha] and TGF-[beta]), EPO receptor (EPO), TPO receptor (TPO), ciliary neurotrophic factor receptor, prolactin receptor, and T-cell receptors.

In addition, hormone receptors may be used, especially for hormones, such as steroidal hormones or protein- or peptide-based hormones, for example, epinephrines, thyroxines, oxytocine, insulin, thyroid-stimulating hormone, calcitonine, chorionic gonadotropine, corticotropine, follicle stimulating hormone, glucagons, leuteinizing hormone, lipotropine, melanocyte-stimulating hormone, norepinephrines, parathyroid hormone, Thyroid-Stimulating Hormone (TSH), vasopressin's, encephalin, serotonin, estradiol, progesterone, testosterone, cortisone, and glucocorticoide. Receptor ligands include those which are on the cell surface receptors of hormones, lipids, proteins, glycol proteins, signal transducers, growth factors, cytokine, and other bio molecules. Moreover, targeting groups can be selected from carbohydrates with the general formula: Cx(H2O)y, wherein herewith also monosaccharides, disaccharides and oligo- as well as polysaccharides are included, as well as other polymers which consist of sugar molecules which contain glycosidic bonds. Carbohydrates may include those in which all or parts of the carbohydrate components contain glycosylated proteins, including the monomers and oligomers of galactose, mannose, fructose, galactosamine, glucosamine, glucose, sialic acid, and the glycosylated components, which make possible the binding to specific receptors, especially cell surface receptors. Other useful carbohydrates include monomers and polymers of glucose, ribose, lactose, raffinose, fructose and other biologically occurring carbohydrates especially polysaccharides, for example, arabinogalactan, gum Arabica, mannan etc., which are suitable for introducing signal generating agents into cells, see U.S. Pat. No. 5,554,386.

Furthermore, targeting groups can include lipids, fats, fatty oils, waxes, phospholipids, glycolipids, terpenes, fatty acids and glycerides, and triglycerides, or eicosanoides, steroids, sterols, suitable compounds of which can also be hormones, such as prostaglandins, opiates and cholesterol etc. All functional groups can be selected as the targeting group, which possess inhibiting properties, such as for example enzyme inhibitors, preferably those which link signal generating agents into/onto enzymes.

Targeting groups can also include functional compounds which enable internalization or incorporation of signal generating agents in the cells, especially in the cytoplasm or in specific cell compartments or organelles, such as, for example, the cell nucleus. For example, such a targeting group may contains all or parts of HIV-1 tat-proteins, their analogs and derivatized or functionally similar proteins, and in this way allows an especially rapid uptake of substances into the cells. As an example, refer to Fawell et al., PNAS USA 91:664 (1994); Frankel et al., Cell 55:1189, (1988); Savion et al., J. Biol. Chem. 256:1149 (1981); Derossi et al., J. Biol. Chem. 269:10444 (1994); and Baldin et al., EMBO J. 9:1511 (1990).

Targeting groups can further include the so-called Nuclear Localisation Signal (NLS), which include positively charged (basic) domains which bind to specifically targeted structures of cell nuclei. Numerous NLS and their amino acid sequences are known including single basic NLS such as that of the SV40 (monkey virus) large T Antigen (pro Lys Lys Lys Arg Lys Val), Kalderon (1984), et al., Cell, 39:499-509), the teinoic acid receptor-[beta] nuclear localization signal (ARRRRP); NFKB p50 (EEVQRKRQKL; Ghosh et al., Cell 62:1019 (1990); NFKB p65 (EEKRKRTYE; Nolan et al., Cell 64:961 (1991), as well as others (see for example Boulikas, J. Cell. Biochem. 55(1):32-58 (1994), and double basic NLS's, such as for example xenopus (African clawed toad) proteins, nucleoplasmin (Ala Val Lys Arg Pro Ala Ala Thr Lys Lys Ala Gly Gln Ala Lys Lys Lys Lys Leu Asp), Dingwall, et al., Cell, 30:449-458, 1982 and Dingwall, et al., J. Cell Biol., 107:641-849, 1988. Numerous localization studies have shown that NLSs, which are built into synthetic peptides which normally do not address the cell nucleus or were coupled to reporter proteins, lead to an enrichment of such proteins and peptides in cell nuclei. Exemplary references are made to Dingwall, and Laskey, Ann, Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990. Targeting groups for the hepatobiliary system may be selected, as suggested in U.S. Pat. Nos. 5,573,752 and 5,582,814.

In exemplary embodiments, the implant comprises absorptive agents, e.g. to remove compounds from body fluids. Suitable absorptive agents include chelating agents, such as penicillamine, methylene tetramine dihydrochloride, EDTA, DMSA or deferoxamine mesylate, any other appropriate chemical modification, antibodies, and micro beads or other materials containing cross linked reagents for absorption of drugs, toxins or other agents.

In some exemplary embodiments, biologically active agents are selected from cells, cell cultures, organized cell cultures, tissues, organs of desired species and non-human organisms.

In exemplary embodiments, the beneficial agents comprise metal based nano-particles that are selected from ferromagnetic or superparamagnetic metals or metal-alloys, either further modified by coating with silanes or any other suitable polymer or not modified, for interstitial hyperthermia or thermoablation.

In another exemplary embodiment, it can be desirable to coat the implant on the outer surface or inner surface with a coating to enhance engraftment or biocompatibility. Such coatings may comprise carbon coatings, metal carbides, metal nitrides, metal oxides e.g. diamond-like carbon or silicon carbide, or pure metal layers of e.g. titanium, using PVD, Sputter-, CVD or similar vapor deposition methods or ion implantation.

In further exemplary embodiments it is preferable to produce a porous coating onto at least one part of the exemplary implant in a further step, such as porous carbon coatings as described in International Publications WO 2004/101177, WO 2004/101017 or WO 2004/105826, or porous composite-coatings as described in International Application PCT/EP2006/063450, or porous metal-based coatings as described in International Publication WO 2006/097503, or any other suitable porous coating.

In further exemplary embodiments a sol/gel-based beneficial agent can be incorporated into the exemplary implant or a sol/gel-based coating that can be dissolvable in physiologic fluids may be applied to at least a part of the implant, as described, e.g. in International Publications WO 2006/077256 or WO 2006/082221.

In some exemplary embodiments, it can be desirable to combine two or more different functional modifications as described above to obtain a functional implant.

Incorporation of beneficial agents may be carried out by any suitable ways, e.g., preferably by dip-coating, spray coating or the like. The beneficial agent may be provided in an appropriate solvent, optionally using additives. The loading of these agents may be carried out under atmospheric, sub-atmospheric pressure or under vacuum. Alternatively, loading may be carried out under high pressure. Incorporation of the beneficial agent may be carried out by applying electrical charge to the implant or exposing at least a portion of the implant to a gaseous material including the gaseous or vapor phase of the solvent in which an agent is dissolved or other gases that have a high degree of solubility in the loading solvent. In preferred embodiments the beneficial agents are provided using carriers that are incorporated into the compartment of the implant. Carriers can be selected from any suitable group of polymers or solvents.

Preferred exemplary carriers can be polymers like biocompatible polymers, for example. In exemplary embodiments, it can be preferable to select carriers from pH-sensitive polymers, like, for example, however not exclusively: poly(acrylic acid) and derivatives, for example: homopolymers like poly(amino carboxylic acid), poly(acrylic acid), poly(methyl acrylic acid) and their copolymers. This applies likewise for polysaccharides like celluloseacetatephthalate, hydroxylpropylmethylcellulosephthalate, hydroxypropylmethylcellulosesuccinate, celluloseacetatetrimellitate and chitosan. In certain embodiments it can be especially preferred to select carriers from temperature sensitive polymers, like for example, however not exclusively: poly(N-isopropylacrylamide-co-sodium-acrylate-co-n-N-alkylacrylamide), poly(N-methyl-N-n-propylacrylamide), poly(N-methyl-N-isopropylacrylamide), poly(N—N-propylmethacrylamide), poly(N-isopropylacrylamide), poly(N,N-diethylacrylamide), poly(N-isopropylmethacrylamide), poly(N-cyclopropylacrylamide), poly(N-ethylacrylamide), poly(N-ethylmethylacrylamide), poly(N-methyl-N-ethylacrylamide), poly(N-cyclopropylacrylamide). Other polymers suitable that can be used as a carrier with thermogel characteristics are hydroxypropylcellulose, methylcellulose, hydroxypropylmethylcellulose, ethylhydroxyethylcellulose and pluronics like F-127, L-122, L-92, L-81, L-61. Exemplary carrier polymers include also, however not exclusively, functionalized styrene, like amino styrene, functionalized dextrane and polyamino acids. Furthermore polyamino acids, (poly-D-amino acids as well as poly-L-amino acids), for example polylysine, and polymers which contain lysine or other suitable amino acids. Other useful polyamino acids are polyglutamic acids, polyaspartic acid, copolymers of lysine and glutamine or aspartic acid, copolymers of lysine with alanine, tyrosine, phenylalanine, serine, tryptophan and/or proline.

Exemplary Methods of Manufacturing

The exemplary implants can be manufactured in one seamless part or with seams from multiple parts. The exemplary implants may be manufactured using known implant manufacturing techniques. Particularly, appropriate manufacturing methods include, but are not limited to, laser cutting, chemical etching or stamping of tubes. Another preferable option is the manufacturing by laser cutting, chemically etching, and stamping flat sheets, rolling of the sheets and, as a further option, welding the sheets. Other appropriate manufacturing techniques include electrode discharge machining or molding the exemplary implant with the desired design. A further option is to weld individual sections together. Any other suitable implant manufacturing process may also be applied and used.

One exemplary option is to use sandwiched tubes or sheets. The sandwich comprises a tube or a sheet of the desired implant material defining the outer and the inner walls, whereby the core of the sandwich comprises any removable or degradable material. The core of the sandwich is referred to as a template for generating the inner compartment or respective reservoir. Removal of templates results in formation of a compartment within the implant. Preferably, the templating material can be removed by using appropriate solvents, particularly if the templating material is an organic compound, a salt or the like. Suitable solvents are for example, (hot) water, diluted or concentrated inorganic or organic acids, bases and the like. Suitable inorganic acids are, for example, hydrochloric acid, sulfuric acid, phosphoric acid, nitric acid as well as diluted hydrofluoric acid. Suitable bases can be, for example, sodium hydroxide, ammonia, carbonate as well as organic amines. Suitable organic acids are, for example, formic acid, acetic acid, trichloromethane acid, trifluoromethane acid, citric acid, tartaric acid, oxalic acid and mixtures thereof.

Exemplary suitable solvents can comprise also, for example, methanol, ethanol, N-propanol, isopropanol, butoxydiglycol, butoxyethanol, butoxyisopropanol, butoxypropanol, n-butyl alcohol, t-butyl alcohol, butylene glycol, butyl octanol, diethylene glycol, dimethoxydiglycol, dimethyl ether, dipropylene glycol, ethoxydiglycol, ethoxyethanol, ethyl hexane diol, glycol, hexane diol, 1,2,6-hexane triol, hexyl alcohol, hexylene glycol, isobutoxy propanol, isopentyl diol, 3-methoxybutanol, methoxydiglycol, methoxyethanol, methoxyisopropanol, methoxymethylbutanol, methoxy PEG-10, methylal, methyl hexyl ether, methyl propane diol, neopentyl glycol, PEG-4, PEG-6, PEG-7, PEG-8, PEG-9, PEG-6-methyl ether, pentylene glycol, PPG-7, PPG-2-buteth-3, PPG-2 butyl ether, PPG-3 butyl ether, PPG-2 methyl ether, PPG-3 methyl ether, PPG-2 propyl ether, propane diol, propylene glycol, propylene glycol butyl ether, propylene glycol propyl ether, tetrahydrofurane, trimethyl hexanol, phenol, benzene, toluene, xylene; as well as water, if necessary in mixture with dispersants, surfactants or other additives and mixtures of the above-named substances. Preferred solvents comprise one or several organic solvents from the group of ethanol, isopropanol, n-propanol, dipropylene glycol methyl ether and butoxyisopropanol (1,2-propylene glycol-n-butyl ether), tetrahydrofurane, phenol, benzene, toluene, xylene, preferably ethanol, isopropanol, n-propanol and/or dipropylene glycol methyl ether, in particular isopropanol and/or n-propanol.

Another exemplary embodiment of the method according to the present invention comprises the thermolytic degradation of the pore-forming material. The temperatures may be in the range of about 100° C. to 1500° C., or in the range of about 300° C. to 800° C. For example, the thermal degradation occurs after manufacturing the desired implant shape using tubes or sheets. It can also be preferred to allow the removal of the templating material by introducing at least one opening.

The templates may be manufactured in the desired shape using conventional implant manufacturing techniques. For example, suitable manufacturing methods may include, but are not limited to, laser cutting, chemical etching, weaving of fibers, stamping of tubes, stamping of flat sheets, rolling of sheets into cylindrical shapes and, as a further option, e.g. welding or gluing of sheets, fibers or other shapes of template material. Other manufacturing techniques include electrode discharge machining or molding the inventive implant with the desired design. A further exemplary option may be to weld or glue individual sections of the template together. Bulk materials may be structured into templates, for example, by folding, embossing, punching, pressing, extruding, gathering, injection molding, and the like. In this way, certain structures of a regular or irregular type may be provided for use as a template according to exemplary embodiments of this invention. Other methods to form a template may include shaping of materials in liquid, pulpy or pasty form, for example, extruding, slip casting, or molding, and hardening the three dimensional template shape, if desired. Other conventional methods to provide templates may include wet or dry spinning methods, electro-spinning and the like, or knitting, weaving and any other known method to produce woven or non-woven articles or forms of regular or irregular shape. For cylindrical or tube-like implant designs, templates may be provided as sheets, foils or tubes, such as sandwiched tubes or sandwiched sheets. The template may be provided in a substantially net shape of the desired implant design.

Exemplary methods for manufacturing the reservoir implants and stents of the exemplary embodiments of the present invention based on metallization of removable templates are described U.S. Provisional application entitled “Medical devices with extended or multiple reservoirs”, which are incorporated herein by reference.

In certain exemplary embodiments, pore sizes and porosity of the walls enclosing the lumen can be controlled by using sol/gel forming metal-based components and a crosslinker. Crosslinkers as additives can be, for example, isocyanates, silanes, diols, di-carboxylic acids, (meth)acrylates, for example, 2-hydroxyethyl methacrylate, propyltrimethoxysilane, 3-(trimethylsilyl)propyl methacrylate, isophorone diisocyanate, polyols, glycerine and the like. Particularly preferred are biocompatible crosslinkers like glycerine, diethylene triamino isocyanate and 1,6-diisocyanato hexane or any other suitable cross-linking agent or any mixture thereof.

A further exemplary embodiment of a manufacturing method according to the present invention can be based in producing ceramic, organic or composite implants according to the present invention. In these embodiments a net shape template may be produced first and a ceramic, an organic or a composite implant by coating of the net shape with the ceramic, organic or composite material. A person having ordinary skill in the art can easily determine the material combination that allows to remove the templating material, e.g., thermolytically or by leaching, without affecting the coating that determines the wall of the implant struts and nodes. In other exemplary embodiments, it may be desirable to manufacture implants using sheets and tubes, whereby the sandwich is composed of a first and a third layer comprising the outer or inner wall of the implant struts or nodes, and a second layer in between comprising the templating material.

It should be noted that the term ‘comprising’ does not exclude other elements or steps and the ‘a’ or ‘an’ does not exclude a plurality. In addition elements described in association with the different embodiments may be combined. It should also be noted that the reference signs in the claims shall not be construed as limiting the scope of the claims.

Having thus described in detail several exemplary embodiments of the present invention, it is to be understood that the present invention described above is not to be limited to particular details set forth in the above description, as many apparent variations thereof are possible without departing from the spirit or scope of the present invention. The exemplary embodiments of the present invention are disclosed herein or are obvious from and encompassed by the detailed description. The detailed description, given by way of example, but not intended to limit the present invention solely to the specific embodiments described, may best be understood in conjunction with the accompanying Figures.

The foregoing applications, and all documents cited therein or during their prosecution (“appln. cited documents”) and all documents cited or referenced in the appln. cited documents, and all documents cited or referenced herein (“herein cited documents”), and all documents cited or referenced in the herein cited documents, together with any manufacturer's instructions, descriptions, product specifications, and product sheets for any products mentioned herein or in any document incorporated by reference herein, are hereby incorporated herein by reference, and may be employed in the practice of the present invention.

What is claimed is:

1. An implantable device or a part thereof, comprising:
a structure having a plurality of walls which enclose a lumen for storing at least one active ingredient, wherein at least one portion of the walls is composed of a material which is biodegradable in-vivo and which facilitates a fluid communication between a lumen and an exterior of the device of the part thereof for releasing the at least one stored ingredient.

2. The device of claim 1, wherein the material is non-porous, and wherein the walls have at least one opening connecting the lumen with the exterior of the device or the part thereof.

3. The device of claim 1, wherein the material is porous having a plurality of interconnected pores.

4. The device of claim 3, wherein the porous material has a porosity determined by pore volume/total volume of material of at least about 10%.

5. The device of claim 2, wherein the at least one opening is a hole.

6. The device of claim 1, wherein the device is a stent.

7. The device of claim 1, wherein the lumen includes the at least one active ingredient.

8. The device of claim 7, wherein the at least one active ingredient is configured to be released in-vivo.

9. The device of claim 7, wherein the at least one active ingredient includes at least one of a pharmacologically, therapeutically, biologically or diagnostically active agent or an absorptive agent.

10. The device of claim 1, wherein the device is a stent structured for maintaining the patency of at least one of the esophagus, trachea, bronchial vessels, arteries, veins, biliary vessels and other similar passageways.

11. The device of claim 1, wherein the material includes at least one of an inorganic material, an organic material, a metal, a ceramic, a polymer or a composite.

12. The device of claim 1, wherein the material includes at least one of a biodegradable or biocorrosive metal, an alloy based on at least one of magnesium or zinc, or an alloy comprising at least one of Mg, Ca, Fe, Zn, Al, W, Ln, Si, or Y.

13. The device of claim 1, wherein the device is expandable from a contracted state suitable for an insertion into a vessel to an expanded state in which the device supports a surrounding tissue.

14. The device of claim 13, wherein the device is self-expandable.

15. The device of claim 1, wherein the lumen has a particular extension in a longitudinal direction of the device and along a circumference of the device, and wherein the particular extension of the lumen is substantially larger than a radial extension of the lumen.

16. The device of claim 1, wherein the device comprises a first tube and a second tube concentric to the first tube, and wherein the lumen is enclosed between the first and second concentric tubes.

17. The device of claim 1, wherein the device comprises a first ribbon helically wound around a tubular space and a second ribbon helically wound around the tubular space corresponding and concentric to the first ribbon.

18. The device of claim 1, wherein the device is formed by a plurality of hollow annular elements each having a sub-lumen, wherein the annular elements are arranged such that each annular element circumferences a tubular space and each annular element has a different inclination from an adjacent abutting annular element, and wherein adjacent ones of the annular elements are joined at an abutting location to form a passage between two abutting ones of the annular elements.

19. The device of claim 18, wherein the annular elements comprise openings facing an exterior of a tubular space.

20. The device of claim 1, wherein the device is formed of a brick wall structured mesh of hollow struts, and wherein continuous struts extend in a longitudinal direction and connected by linking struts.

21. The device of claim 20, wherein the brick walled structure completely circumferences a tubular space, such that the brick walled structure repeats periodically and perpetually along the circumference.

22. The device of claim 1, wherein the device is formed by a plurality of hollow annular wave elements each having a sub-lumen, wherein the annular wave elements are arranged such that each of the annular elements circumferences a tubular space and each of the annular element abutting an adjacent annular element, and wherein adjacent annular elements are joined at an abutting location to form a passage between two abutting ones of the annular elements.

23. The device of claim 22, wherein the tubular space has a shape of a bifurcated tube.
